(19) United States
(12) Patent Application Publication
(10) Pub. No.: US 2026/0327576 A1
(43) Pub. Date: Oct. 22, 2026

(54) METHOD OF ATTENUATING FLAVIVIRUSES

(71) Applicant: National University of Singapore, Singapore (SG)

(72) Inventors: Jang Hann CHU, Singapore (SG); Wei-Xin CHIN, Singapore (SG)

(73) Assignee: National University of Singapore, Singapore (SG)

(21) Appl. No.: 19/113,985

(22) PCT Filed: Aug. 15, 2023
(86) PCT No.: PCT/SG2023/050560

§ 371 Date: Mar. 21, 2025

(30) Foreign Application Priority Data
Sep. 23, 2022 (SG) 10202251123G

(51) Int. Cl. C12N 7/04

(52) U.S. Cl. CPC C12N 7/04; C12N 2770/24121; C12N 2770/24122; C12N 2770/24162

(57) ABSTRACT

The invention relates generally to virology. In particular, the specification teaches a method of attenuating a flavivirus, by modifying the viral genome to have a protein coding sequence having one or more synonymous codons as compared to the protein coding sequence of a parent virus, wherein the modified viral genome is associated with increased protein expression as compared to the viral genome of the parent virus in a mammalian host. Provided herein is also a modified flavivirus as defined herein.

### FIELD OF INVENTION

The invention relates generally to virology. In particular, the specification teaches a method of attenuating a flavivirus. Provided herein is also a modified flavivirus as defined herein.

### BACKGROUND

Synonymous virus genome recoding is an approach to rationally designing live attenuated vaccines. Virus genome recoding is an attenuation method that involves rewriting a virus genome with many silent mutations. The recoded viral genes still encode for the same protein sequence, which means the viral antigens are not affected. Instead, recoding typically alters the frequencies of favourable or unfavourable codons or the frequencies of CpG or UpA dinucleotides. Recoding typically attenuates a virus by increasing the frequency of unfavourable codons, or unfavourable dinucleotide sequences, or some combination of both. In other words, attenuation is performed by de-optimising the virus genome.

Recoding is an attractive attenuation method because it has the potential to create vaccine strains with exceptional genetic stability. This is because recoding introduces a lot of attenuating mutations, each with a very minor attenuating effect. This means that there is very little selection pressure for an individual mutation to revert, because the fitness advantage of a single reversion mutation would be barely noticeable.

However, previous attempts at developing attenuated flavivirus strains using de-optimisation approaches demonstrated inconsistent results. Furthermore, they have not always been successful at attenuating viral replication in mosquitos. This is a major shortcoming because live attenuated flavivirus vaccines must lack transmissibility in mosquitoes.

Accordingly, it is generally desirable to overcome or ameliorate one or more of the above mentioned difficulties.

### SUMMARY

Disclosed herein is a method of attenuating a flavivirus, the method comprising modifying the viral genome to have a protein coding sequence having one or more synonymous codons as compared to the protein coding sequence of a parent virus, wherein the modified viral genome is associated with increased protein expression as compared to the viral genome of the parent virus in a mammalian host.

Disclosed herein is a modified flavivirus obtained according to the method as defined herein.

Disclosed herein is a modified flavivirus comprising a viral genome comprising a modified virus protein coding sequence having one or more synonymous codons as compared to the protein coding sequence of a parent virus, wherein the modified viral genome is associated with increased protein expression as compared to the viral genome of the parent virus in a mammalian host.

Disclosed herein is an isolated polynucleotide comprising a nucleic acid sequence encoding a modified flavivirus as defined herein.

Disclosed herein is an expression construct comprising a polynucleotide as defined herein.

Disclosed herein is a vector comprising a polynucleotide as defined herein.

Disclosed herein is an immunogenic composition comprising a modified flavivirus or a vector as defined herein.

Disclosed herein is a modified flavivirus, a vector or an immunogenic composition as defined herein for use as a medicament or vaccine.

Disclosed herein is a method of modulating an immune response in a subject, the method comprising administering a therapeutically effective amount of a modified flavivirus, a vector or an immunogenic composition as defined herein to the subject.

Disclosed herein is a method of preventing or treating a flaviviral infection in a subject, the method comprising administering a therapeutically effective amount of a modified flavivirus, a vector or an immunogenic composition as defined herein to the subject.

### BRIEF DESCRIPTION OF DRAWINGS

Embodiments of the present invention are hereafter described, by way of non-limiting example only, with reference to the accompanying drawings in which:

FIG. 1. Virus recoding with silent mutations. (a) The flavivirus genome contains many functional RNA elements such as RNA secondary structures. (b) Recoding is an attenuation method that rewrites large portions of the virus genome with silent mutations. These silent mutations change the nucleotide sequence, but the encoded protein sequence remains the same. Therefore, both protein function and antigen sequences remain unchanged. However, the silent mutations abrogate the sequence and function of RNA elements which play important roles in regulating the virus replication cycle.

FIG. 2. Initial characterisation of DENV2 genome recoding. DENV2-GFP is a dengue reporter virus that expresses EGFP. (a) Genomic maps showing regions of the DENV2-GFP genome recoded with silent mutations. (b) & (c) Fluorescent microscopy analysis of BHK-21 cells infected with recoded DENV2-GFP (10× magnification). Green fluorescent signal indicates DENV2-GFP infected cells. Mock: mock infected control cells. WT: cells infected with wildtype (non-recoded) DENV2-GFP. (b) rcE2-90 and rcE2 clones: partial recoding of Env coding region. (c) rcNS1 clone: recoding of NS1 coding region, with partial overlap into Env and NS2A coding regions.

FIG. 3. Characterisation of recoded DENV2 protein translation and RNA replication efficiency. DENV2 FLuc translation reporter construct expresses Firefly luciferase as a reporter of viral protein translation efficiency. It cannot undergo RNA replication because it contains a deletion of the NS5 protein GDD catalytic triad. DENV2 ΔCap Replicon is a subgenomic DENV2 replicon that is able to undergo viral RNA replication. However it cannot undergo packaging due to a deletion in the capsid coding region. (b) Firefly luciferase assay was used to measure viral protein translation efficiency of non-recoded and recoded DENV2 FLuc constructs. (c) qRT-PCR was used to measure viral RNA levels to compare the viral RNA replication efficiency of non-recoded and recoded DENV2 replicons. *: p-value of <0.05. **: p-value of <0.01. ***: p-value of <0.001. (d) Viral growth kinetics of wildtype (non-recoded) and recoded DENV2-EGFP in BHK-21 cells. Viral titres were measured using fluorescent focus formation assay. FFU: fluorescent focus forming units.

FIG. 4. Characterising effects of increasing degrees of recoding in DENV2. DENV2-GFP is a dengue reporter virus that expresses EGFP. (a) Genomic maps showing regions of the DENV2-GFP genome recoded with silent mutations. (b) & (c) Fluorescent microscopy analysis of BHK-21 cells infected with recoded DENV2-GFP (10× magnification). Green fluorescent signal indicates DENV2-GFP infected cells. Mock: mock infected control cells. WT: cells infected with wildtype (non-recoded) DENV2-GFP.

FIG. 5. Recoded DENV2 clones have reduced replication efficiency. (a) Genomic maps showing regions of the DENV2 genome recoded with silent mutations. (b) to (e) BHK-21 hamster kidney cells, Huh-7 human hepatocarcinoma cells, HepG2 hepatoma cells, and C6/36 Aedes mosquito cells were infected with wildtype or recoded DENV2 clones (rcCap-prM, rcCap-Env, and rcCap-NS1) at an MOI of 0.02. Viral titres were measured using plaque assay. N.D.: virus titre was below the limits of detection for our plaque assay (10 PFU/ml).

FIG. 6. Recoded ZIKV clones have reduced replication efficiency. (a) Genomic maps showing regions of the ZIKV genome recoded with silent mutations. (b) BHK-21 hamster kidney cells, Huh-7 human hepatocarcinoma cells, Vero E6 monkey kidney cells, and C6/36 Aedes mosquito cells were infected with wildtype or recoded ZIKV clones (rcprM-NS5, rcCap-NS3, and rcCap-NS5) at an MOI of 0.01. Viral titres were measured using plaque assay. N.D.: virus titre was below the limits of detection for our plaque assay (10 PFU/ml).

FIG. 7. Small plaque phenotype of recoded DENV2 and ZIKV. (a) Plaques formed by wildtype and recoded ZIKV clones (rcprM-NS5, rcCap-NS3, and rcCap-NS5). (b) & (c) Changes in plaque sizes of wildtype and recoded DENV2 clones (rcCap-prM, rcCap-Env, and rcCap-NS1) during serial passage in BHK-21 cells. (c) Plaque sizes were measured in ImageJ using ViralPlaque Fiji macro. *: p-value of <0.05. ***: p-value of <0.001.

FIG. 8. Predicted structure of DCS-PK RNA element in wildtype and recoded DENV2 clones. Capital letters indicate the original nucleotide sequence. Small letters indicate nucleotide substitutions. (a) Predicted structure for Wildtype DENV2. (b) Predicted structure for recoded DENV2 clones (rcCap-prM, rcCap-Env, and rcCap-NS1). Arrows indicate the positions of silent mutations introduced during codon optimisation: a177g in stem 2, as well as u196a and c197g in stem 3. (c) Predicted structure for rcCap-Env with the predominant a158u and u173c rescue mutations (indicated by the arrows). (d) Predicted structure of rcCap-NS1 with the predominant a158u and c181u rescue mutations (indicated by the arrows).

FIG. 9. Small plaque phenotype of recoded and rescue mutants of DENV2. (a) Plaques formed by wildtype DENV2, wildtype DENV2 with Env-M196V cell line adaptation mutation (WT+rsEnv), recoded DENV2 (rcCap-Env), and rescue mutant of DENV2-rcCap-Env (+rsCE, +rsCap, and +rsEnv). (b) Plaque sizes were measured in ImageJ using ViralPlaque Fiji macro. *: p-value of <0.05. ****: p-value of <0.0001. (c) Recoded ZIKV clones are viable. Recoding of the capsid coding region confers a small plaque phenotype. ***: p-value of <0.001.

FIG. 10. Recoded DENV2 has disrupted RNA structural elements and genetically stable attenuation. (a) RNA SHAPE-Map analysis of DENV2 genomic RNA structures. Analysis was performed on BHK-21 cells infected with either wildtype DENV2 or the rcCap-Env+rsCap recoded rescue clone. After infection, NAI treatment was performed to modify single-stranded nucleotides, after which total RNA extraction, cDNA library preparation and Illumina sequencing was performed according to the SHAPE-Map protocol. Differences in SHAPE were assessed using ΔSHAPE. Inverted triangle indicates location of mutations found in the rcCap-Env+rsCE clone. Positive and negative changes to reactivity compared to wildtype DENV2 are indicated in blue and purple respectively. Positive changes represent reduced reactivity, indicating increased base pairing by a particular nucleotide. (b) Predicted RNA secondary structures in the 5′UTR and structural protein coding region of wildtype DENV2-16681 and rcCap-Env+rsCap rescue clone. In silico modelling of DENV2 genomic RNA secondary structures was performed using the Superfold pipeline with RNAstructure v6.3 as the backend, with the results of our SHAPE analysis incorporated as a constraint. RNA structures were then visualized using VARNA 3.93 and a custom script to map SHAPE reactivity data onto the resulting figure. (c) Rescue clones of recoded DENV2-rcCap-Env retain attenuated growth kinetics. BHK-21 hamster kidney cells and Huh-7 human hepatocarcinoma cells were infected with wildtype DENV2, wildtype DENV2 with Env-M196V cell line adaptation mutation (WT+rsEnv), recoded DENV2 (rcCap-Env), and rescue mutants of DENV2-rcCap-Env (rcCap-Env+rsCE, +rsCap, and +rsEnv) at an MOI of 0.1. Viral titres were measured using plaque assay. Limit of detection for our plaque assay is 10 PFU/ml. (d) Recoded and rescue clones of DENV2 demonstrate attenuation of neurovirulence in suckling mice. Newborn outbred white ICR mice that were less than 24 hours old were challenged by intracranial inoculation with wildtype DENV2, WT+rsEnv, rcCap-Env, or rc+rsCE clones at a dose of 100 PFU per mouse. The mice were kept for four weeks and observed daily for clinical symptoms. Mice that reached a humane endpoint were euthanized. Group sizes: PBS control, n=10; wildtype DENV2, n=11; WT+rsEnv, n=12; rcCap-Env, n=9; rCap-Env+rsCE clone, n=11; rcCap-Env+rsCap, n=10; rcCap-Env+rsEnv, n=9. **: p-value of <0.01. ***: p-value of <0.001. (e) Recoded DENV2 demonstrates attenuation in its Aedes albopictus mosquito vector. Aedes albopictus mosquitoes were fed an infectious blood meal containing 2.5×10⁷ PFU/ml of either wildtype DENV2 or DENV2-rcCap-Env+rsCE. At 11 days post challenge, the mosquitoes were harvested and their infection status and viral loads were determined using plaque assay. Group sizes for both were n=30. Limit of detection is 100 PFU/ml. ***: p-value of <0.001. (d) Recoded ZIKV clones demonstrate attenuation of neurovirulence in suckling mice. Newborn outbred white ICR mice that were less than 24 hours old were challenged by intracranial inoculation with wildtype ZIKV, ZIKV-rcCap-NS3, or ZIKV-rcCap-NS5. The mice were kept for four weeks and observed daily for clinical symptoms. Mice that reached a humane endpoint were euthanized. Group sizes: PBS control, n=10; wildtype ZIKV 10 PFU/ml, n=11; wildtype ZIKV 100 PFU/ml, n=12; wildtype ZIKV 103 PFU/ml, n=11; rcCap-NS3 10 PFU/ml, n=15; rcCap-NS3 100 PFU/ml, n=10; rcCap-NS3 10³ PFU/ml, n=11; rcCap-NS5 100 PFU/ml, n=12; rcCap-NS5 10³ PFU/ml, n=14.

FIG. 11. Genomic maps showing regions of the flavivirus genome that correspond to potential recoded modules. Module 1 corresponds to most of the capsid coding region, starting from the 76th nucleotide (26th codon). This excludes the first 75 nucleotides of the capsid coding region. Recoded module 1 contributes the most to the loss of replication efficiency or even loss of viability in mosquito cells. Module 2 corresponds to the premembrane and envelope coding regions. Recoded module 1 and 2 have the greatest contribution to the loss of replication efficiency in mammalian and human cells. Furthermore, recoded modules 1 and 2 by themselves are sufficient for in vivo attenuation in mice. Module 3 corresponds to the NS1, NS2A, and NS2B coding region, as well as the first half of the NS3 coding region. Module 4 corresponds to the latter half of the NS3 coding region, as well as the NS4A, NS4B, and NS5 coding regions. Recoded modules 3 and 4 do have a measurable impact on virus replication efficiency. However, their contribution to the overall degree of attenuation is not as strong as recoded module 1 and 2.

FIG. 12. Predicted structure of DCS-PK RNA element in wildtype and recoded flavivirus clones. (a) Wildtype DENV1. (b) Recoded DENV1. (c) Wildtype DENV3. (d) Recoded DENV3.

FIG. 13. Predicted structure of DCS-PK RNA element in wildtype and recoded flavivirus clones. (a) Wildtype DENV4. (b) Recoded DENV4. (c) Wildtype KUNV. (d) Recoded KUNV.

FIG. 14. Predicted structure of DCS-PK RNA element in wildtype and recoded flavivirus clones. (a) Wildtype ZIKV. (b) Recoded ZIKV.

FIG. 15. Characterisation of putative DENV2 envelop stem RNA element (ESRE). (a) Genomic maps showing regions of the DENV2-EGFP genome recoded with silent mutations. The recoding targets a segment near the 3′ end of the Env protein coding region. The number at the end of each clone indicates the number of codons targeted for recoding, (b) Fluorescent microscopy analysis of BHK-21 cells infected with recoded DENV2-GFP at 4 days post infection (10× magnification). (c) Predicted RNA secondary structure of putative ESRE in wildtype DENV2-16681. Black arrows indicate the nucleotides that are mutated in the rcE2-50 recoded clone.

FIG. 16. Genomic maps of rescue mutants for wildtype DENV2 and DENV2-rcCap-Env. Approximate location of recapitulatory rescue mutations are shown above the genome. Approximate region of recoding is shown below the genome.

FIG. 17. The NS1-G53D mutation is a broad-spectrum attenuating mutation for the mosquito-borne flaviviruses. (a) NS1-G53 is a conserved amino acid residue that is found across the mosquito-borne flaviviruses. (b) & (c) The NS1-G53D mutation was cloned into recoded virus backbones. Viral replication efficiency was investigated by infecting human and mammalian cells with recoded virus with or without the NS1-G53D mutation. (b) Viral titres in ZIKV-infected cells over five days. (c) Viral titres in DENV2- or DENV4-infected cells over five days.

FIG. 18. NS1-G53D is a broad-spectrum immunogenic mutation for the mosquito-borne flaviviruses. The immunogenicity of wildtype ZIKV and DENV4 virus was compared against a recoded virus backbone carrying the NS1-G53D mutation. Mock infected cells was used as the control. A549 cells were infected and harvested after 24 hours for analysis. qRT-PCR analysis was performed to determine the early interferon response. The Log2 fold-change induction of interferon genes is relative to the mock control.

FIG. 19. Genetic stability of the NS1-G53 ATG and TCA mutations. The ATG and TCA mutations encode for the attenuating Met and Ser respectively. Further, any single nucleotide substitution that can be introduced into ATG or TCA will result in a secondary codon that encodes for an attenuating amino acid, a lethal amino acid, or a stop codon.

### DETAILED DESCRIPTION

The present specification is directed to a method of attenuating a flavivirus, the method comprising modifying the viral genome to have a protein coding sequence having one or more synonymous codons as compared to the protein coding sequence of a parent virus, wherein the modified viral genome is associated with increased protein expression as compared to the viral genome of the parent virus in a mammalian host.

Previous attempts at attenuating flaviviruses through codon replacement have focused on codon de-optimization to reduce protein expression in a mammalian host, e.g., by replacing the codons of a parent virus with synonymous codons that are less frequently or rarely used in the host. The inventors have made the remarkable discovery that codon optimization of a flaviviral genome can also lead to viral attenuation.

Without being bound by theory, the synonymous codons introduced into the flavivirus genome during codon optimization can abrogate the sequence and function of RNA elements (e.g., pseudoknots and other secondary RNA structures) that are essential for efficient viral replication. These RNA elements can regulate viral replication by regulating the transition from the viral protein expression state to the viral RNA replication state. By disrupting these RNA elements, the genome recoding method of the present disclosure can inhibit or delay the transition from the viral protein expression state to the viral RNA replication state. This means that the virus remains in the protein expression state for longer, leading to both an increase in viral protein expression and a reduction in viral replication efficiency. By spacing out the synonymous codons across stretches of the flaviviral protein coding sequence, a number of these RNA elements may be disrupted without prior knowledge of the RNA structures in the flavivirus. The cumulative effect of many of these disruptive mutations can reduce viral RNA replication efficiency to such an extent that the virus is attenuated, despite viral protein production being enhanced in a host. The inventors have found that the increase in viral protein expression from genome recoding is a linear increase. In contrast, the decrease in viral RNA replication from recoding is an exponential decrease. This exponential decrease in viral RNA replication can outweigh any linear increase in viral protein expression, leading to a decrease in the overall viral replication efficiency.

This method of viral attenuation, also referred to herein as “optimized recoding”, can be scaled to provide various degrees of attenuation, with a larger extent of recoding correlating with a greater reduction in replication efficiency. Since viral protein sequences are unaffected, the attenuated viruses are likely to retain immunogenicity and can be used for vaccine development. The method disclosed herein can also be combined with other attenuation methods to increase attenuation of under-attenuated vaccine candidates.

As used herein, the term “attenuated virus” or “live attenuated virus” refers to a virus that is altered from an original parental or wild-type virus in such a way that it has a diminished capacity to infect a host, replicate within a host and/or be packaged for re-infection. The virus may be attenuated in a single host, in several hosts or in all its hosts. Such hosts may be any natural or capable host of the virus and includes, for example, mammalian and non-mammalian hosts. Compared with a wild-type virus, an attenuated virus demonstrates substantially reduced or preferably no clinical signs of disease when administered to a subject, while retaining the ability to induce an immune response similar to the wild-type virus.

Viral attenuation can be confirmed in ways that are well known to one of ordinary skill in the art. Non-limiting examples include plaque assays, cell viability assays, reduced viral load in a host tissue or organism, and reduced morbidity or lethality in test animals.

As used herein, the term “attenuated replication”, in the context of a virus, refers to a reduction in the capacity of the virus to replicate or a reduction in the rate of viral replication, as compared to a wildtype or parent virus. Attenuation of viral replication can be measured by any techniques used to measure viral replication in the art, such as by viral yield or titre, or by the rate of viral replication or production. For example, the reduction in replication can be a reduction in the number of plaque forming units, in the number of virions, in the amount of viral genomic material, etc.

The terms “parent virus” and “parent protein encoding sequence” are used herein to refer to viral genomes and protein encoding sequences from which new sequences are derived. Parent viruses and sequences are usually “wildtype” or “naturally occurring” prototypes or isolates of variants for which it is desired to obtain an attenuated virus. However, parent viruses also include mutants specifically created or selected in the laboratory on the basis of real or perceived desirable properties. Accordingly, parent viruses that are candidates for attenuation include mutants of wildtype or naturally occurring viruses that have nucleotide or amino acid deletions, insertions, substitutions and the like, and also include mutants which have codon substitutions.

As used herein, the term “attenuated vaccine” or “live attenuated vaccine” refers to a pharmaceutical composition containing a live attenuated pathogen, such as a virus. The pharmaceutical composition contains at least one immunologically active component that induces an immune response in a subject against a virus, and optionally can include one or more additional components that enhance the immunological activity of the active component. An attenuated vaccine can additionally include further components typical to pharmaceutical compositions. The at least one immunologically active component is one or more of the attenuated viruses defined herein.

As used herein, it will be understood that the term “flavivirus” encompasses all viruses within the Flaviviridae family as classified under the Baltimore Classification System (BCS) and/or the International Committee on Taxonomy of Viruses (ICTV). Non-limiting examples of suitable flaviviruses include the following: Alfuy virus, Aroa virus, Bagaza virus, Banzi virus, Bouboui virus, Bussuquara virus, Cacipacore virus, Dengue virus (e.g., Dengue virus serotype I, Dengue virus serotype II, Dengue virus serotype III, Dengue virus serotype IV), Edge Hill virus, Gadgets Gully virus, Kadam virus, Kunjin virus, Kokobera virus, Kyasanur Forest disease virus, Iguape virus, Ilheus virus, Israel turkey meningoencephalomyelitis virus, Japanese encephalitis virus (JEV), Jugra virus, Kedougou virus, Koutango virus, Langat virus, Louping ill virus, Meaban virus, Modoc Virus, Murray Valley encephalitis virus (MVEV), Naranjal virus, Negeishi Virus, Ntaya virus, Omsk hemorrhagic fever virus, Potiskum virus, Powassan virus, Rio Bravo Virus, Rocio virus, Russian Spring Summer Encephalitis Virus, Royal Farm virus, Saboya virus, Saumarez Reef virus, Sepik virus, St. Louis encephalitis virus, Spondweni virus, Stratford virus, Tick-borne encephalitis virus, Tembusu virus, Tyuleniy virus, Uganda S virus, Usutu virus, Wesselsbron virus, West Nile virus (WNV), Yellow fever virus (YFV), Yaounde virus, and Zika virus.

The flavivirus genome is a positive-sense, single-stranded RNA genome. It contains a single open reading frame (ORF), flanked by 5′- and 3′-untranslated or non-coding regions. The ORF encodes a single polypeptide comprising three structural proteins [capsid (C), premembrane (prM), and Envelope (E)] and at least seven non-structural (NS) proteins (NS1, NS2A, NS2B, NS3, NS4A, NS4B, NS5). The polypeptide is co- or post-translationally cleaved to give the component proteins.

As used herein, the term “host” refers, in the context of a virus, to any host organism or part of a host organism (e.g., a cell, tissue, organ or organ system) that is capable of being infected with and propagating the virus. A host may be a mammalian host or a non-mammalian host (e.g., an arthropod, bird or shell fish).

“Nucleic acid” as used herein includes “polynucleotide”, “oligonucleotide”, and “nucleic acid molecule”, and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

“Polynucleotide sequence” as used herein can refer to the polynucleotide material itself and/or to the sequence information (e.g., the succession of letters used as abbreviations for bases) that biochemically characterizes a specific nucleic acid. A polynucleotide sequence presented herein is presented in a 5′ to 3′ direction unless otherwise indicated.

“Polypeptide”, “peptide” or “protein” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

The term “isolated” refers to a biological material, such as a virus, a nucleic acid or a polypeptide, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native to a material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated virus, for example, is in an environment (e.g., a cell culture system, or purified from cell culture) other than the native environment of wild-type virus (e.g., in an infected individual).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include an RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

By “coding sequence” or “protein coding sequence” is meant any nucleic acid sequence that contributes to the code for the polypeptide product of a gene. By contrast, the term “non-coding sequence” refers to any nucleic acid sequence that does not contribute to the code for the polypeptide product of a gene. Thus, a “protein coding sequence” of a flavivirus may be any nucleic acid sequence derived from the viral genome that is part of the coding sequence of a polypeptide. The nucleic acid sequence may be DNA or RNA. The protein coding sequence may be, for example, the flaviviral ORF or a portion of the flaviviral ORF. For the avoidance of doubt, it is not necessary for a protein coding sequence of this disclosure to be the full-length coding sequence of a polypeptide, or for the protein coding sequence to begin or end with a complete codon.

The term “protein expression” herein, with respect to a gene sequence, refers to the processes of transcription and translation to produce a protein from the gene. “Protein expression”, with respect to a flaviviral genome, refers to the process of translating viral RNA to a protein.

As used herein, the term “5′ untranslated region” or “5′ UTR” refers to a sequence located 3′ to a promoter region and 5′ of the downstream coding sequence. Thus, such a sequence, while transcribed, is upstream (i.e., 5′) of the translation initiation codon and therefore is generally not translated into a portion of the polypeptide product. The term “3′ untranslated region” or “3′ UTR” refers to a nucleotide sequence downstream (i.e., 3′) of a coding sequence. It extends from the first nucleotide after the stop codon of a coding sequence to just before the poly(A) tail of the corresponding transcribed mRNA. The 3′ UTR may contain sequences that regulate.

The term “promoter”, as used herein, refers to a DNA sequence that determines the site of transcription initiation for an RNA polymerase. Promoter sequences comprise motifs which are recognized and bound by polypeptides, i.e. transcription factors. The transcription factors recruit RNA polymerase upon binding RNA polymerases II, preferably, RNA polymerase I, II or III, more preferably, RNA polymerase II or III, and most preferably, RNA polymerase II. Thereby will be initiated the transcription of a nucleic acid operatively linked to the transcription control sequence. It is to be understood that dependent on the type of nucleic acid to be expressed, expression as meant herein may comprise transcription of DNA sequences into RNA polynucleotides (as suitable for, e.g., anti-sense approaches, RNAi approaches or ribozyme approaches) or may comprise transcription of DNA sequences into RNA polynucleotides followed by translation of the said RNA polynucleotides into polypeptides (as suitable for, e.g., gene expression and recombinant polypeptide production approaches). In one embodiment, the transcription control sequence is located immediately adjacent to the nucleic acid to be expressed, i.e. physically linked to the said nucleic acid at its 5′ end.

The term “synonymous codons” refers to two or more nucleotide codons that encode the same amino acid. Indeed, most amino acids are encoded by more than one codon (see the genetic code in Table 1). For instance, alanine is encoded by four synonymous codons GCU, GCC, GCA, and GCG. Three amino acids (Leu, Ser, and Arg) are encoded by six synonymous codons, while only Trp and Met have unique codons.

Synonymous codons are not used with equal frequency. The preference or variation in codon usage is also known as codon usage bias or codon bias, and varies by organism. In general, the most frequently used codons in a particular organism are those for which the cognate tRNA is abundant, and the use of these codons may enhance the rate and/or accuracy of protein translation. Conversely, tRNAs for rarely used codons are found at relatively low levels, and the use of these codons is thought to reduce translation rate and/or accuracy.

Codons that are used at a significantly lower frequency in a particular organism are also called “rare” codons. For instance, the following codons are considered rare in humans: GCG (Ala), CCG (Pro), CGU (Arg), CGC (Arg), UCG (Ser), and ACG (Thr).

TABLE 1
Synonymous codons

| | Three | One | Synonymous |
|---|---|---|---|
| Amino acid | letter code | letter symbol | codons |
| Alanine | Ala | A | GCU, GCC, GCA, GCG |
| Arginine | Arg | R | AGA, AGG, CGU, CGC, |
| | | | CGA, CGG |
| Asparagine | Asn | N | AAU, AAC |
| Aspartic acid | Asp | D | GAU, GAC |
| Cysteine | Cys | C | UGU, UGC |
| Glutamine | Gln | Q | CAA, CAG |
| Gliutamic acid | Glu | E | GAA, GAG |
| Glycine | Gly | G | GGU, GGC, GGA, GGG |
| Histidine | His | H | CAU, CAC |
| Isoleucine | Ile | I | AUU, AUC, AUA |
| Leucine | Leu | L | UUA, UUG, CUU, CUC, |
| | | | CUA, CUG |
| Lysine | Lys | K | AAA, AAG |
| Methionine | Met | M | AUG |
| Phenylalanine | Phe | F | UUU, UUC |
| Proline | Pro | P | CCU, CCC, CCA, CCG |
| Serine | Ser | S | UCU, UCC, UCA, AGU, |
| | | | AGC |
| Threonine | Thr | T | ACU, ACC, ACA, ACG |
| Tryptophan | Trp | W | UGG |
| Tyrosine | Tyr | Y | UAU, UAC |
| Valine | Val | V | GUU, GUC, GUA, GUG |
| STOP | — | — | UAA, UAG, UGA |

The methods herein comprise substituting codons in a wiidtype or parent virus protein coding sequence with synonymous codons so that viral protein expression is increased in a mammalian host. The selection of codons to substitute and the choice of synonymous codons for substitution may be guided by general principles understood by a skilled person. For instance, viral codons with rare or low usage frequency in a mammalian host may be replaced with codons that are more frequently used such that viral protein expression is increased in the mammalian host. A skilled person may refer to various commonly used databases for codon usage tables specific to particular hosts. One such database is accessible at http://www.kazusa.or.jp/codon. Codons may be replaced to, for example, remove a continuous stretch of the same codon or a continuous stretch of the same nucleotide such that the protein coding sequence gives increased viral protein expression in the mammalian host. This may require replacing a codon with higher usage frequency with a less frequently use codon. Codons may also be replaced to remove predicted mRNA secondary structures. This may also require replacing a more frequently used codon with a less frequently used one. In some instances, rare codons may be retained, for example, to avoid introducing predicted RNA secondary structures into the coding sequence. Synonymous codon substitution may occur at random positions within a protein coding sequence. Various tools are available for codon optimization of a target coding sequence in mammalian hosts, including tools from Genscript, Integrated DNA Technologies, VectorBuilder, NovoPro and Genewiz Azenta.

Viral protein expression may be measured using any method known in the art for quantifying protein levels, including but not limited to immunological methods (e.g., Western analysis, enzyme-linked immunosorbent assay, immunoprecipitation or immunofluorescence staining); detection with labelled aptamers, lectins or other molecules that bind to a viral protein; mass spectrometry; and protein activity or functional assays. The parent virus may be engineered to express a heterologous protein (e.g., a fluorescent or colorimetric protein or an enzyme) whose level is quantified to determine viral protein expression. A non-replicating parent virus may be used to measure viral protein expression independent of viral replication.

A modified viral genome having one or more synonymous codons may be associated with increased (or optimized) protein expression when the one or more synonymous codons is predicted or found to lead to increased protein expression as compared to the viral genome of the parent virus in a mammalian host.

The modified viral genome may be associated with an increase in protein expression that is at least about 10% compared to the parent viral genome in a mammalian host. The increase in protein expression from the modified viral genome may be at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% higher than protein expression from the parent virus genome in a mammalian host.

In one embodiment, the modified viral genome is also associated with decreased protein expression in a non-mammalian host as compared to the viral genome of the parent virus.

The modified viral genome may be associated with a decrease in protein expression that is at least about 10% compared to the parent viral genome in a non-mammalian host. The decrease in protein expression from the modified viral genome may be at least about 10%, at least about 20%, at least about 30%, at least about 40%, at least about 50%, at least about 60%, at least about 70%, at least about 80%, or at least about 90% lower than protein expression from the parent virus genome in a non-mammalian host.

In one embodiment, the mammalian host is a human. In one embodiment, the non-mammalian host is an arthropod, bird or shell fish, preferably a mosquito or tick.

In one embodiment, the method attenuates replication of the flavivirus in both the mammalian host and the non-mammalian host. The replication of the modified flavivirus may be reduced by at least about 10%, at least about 20%, at least about 30%, at least about 40%, at least about 50%, at least about 60%, at least about 70%, at least about 80%, at least about 90% or up to 100% as compared to the parent flavivirus in the mammalian host and/or in the non-mammalian host.

In some embodiments, the one or more synonymous codons disrupts at least one RNA element involved in viral replication. RNA elements involved in viral replications include but are not limited to pseudoknots, RNA secondary structures (e.g., hairpins, helices, etc.), cyclization sequences, and long-range RNA interactions that help to regulate viral protein expression or RNA replication. Examples of flaviviral RNA elements are described in Liu et al, Rev Med Virol 2019; e2092 (doi: 10.1002/rmv.2092) and Ramos-Lorente et al, Int J Mol Sci 2021, 22(7), 3738 (doi: 10.3390/ijms22073738), which are incorporated by reference herein.

In one embodiment, the at least one RNA element comprises the downstream of 5′ cyclization sequence pseudoknot (DCS-PK). The DCS-PK RNA motif is a three-stem pseudoknot located in the flaviviral capsid coding region. The motif may enhance viral replication by facilitating genome cyclization.

In some embodiments, the method modifies the viral genome to have a protein coding sequence having at least about 50% of synonymous codons. The protein coding sequence may be modified to have at least about 50%, at least about 55%, at least about 60%, at least about 65%, at least about 70%, at least about 75%, at least about 80%, at least about 85%, at least about 90%, or at least about 95% synonymous codons.

In one embodiment, the method modifies the viral genome to have a protein coding sequence of about 60% of synonymous codons.

In one embodiment, the protein coding sequence to be modified is the flaviviral open reading frame (ORF), and the method modifies the flaviviral ORF to have at least about 20% of synonymous codons. The ORF may be modified to have at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of synonymous codons.

In one embodiment, the method modifies the viral genome to have a viral ORF of about 60% of synonymous codons.

In some embodiments, the synonymous codons are randomly distributed within the protein coding sequence or a portion of the protein coding sequence.

In some embodiments, the synonymous codons are randomly distributed within the portion of the protein coding sequence that comprises or consists of the coding sequence or a fragment thereof of the capsid protein (C), premembrane protein (prM), envelop protein (E), non-structural (NS) protein 1 (NS1), NS2A protein, NS2B protein, NS3 protein, NS4A protein, NS4B protein or NS5 protein, or a combination thereof.

In some embodiments, the synonymous codons are randomly distributed within the portion of the protein coding sequence that comprises or consists of: the C coding sequence or a fragment thereof; the C, prM and E coding sequences, or a fragment thereof; the C, prM, E, NS1, NS2A, NS2B and NS3 coding sequences, or a fragment thereof; or the C, prM, E, NS1, NS2A, NS2B, NS3, NS4A, NS4B and NS5 coding regions, or a fragment thereof.

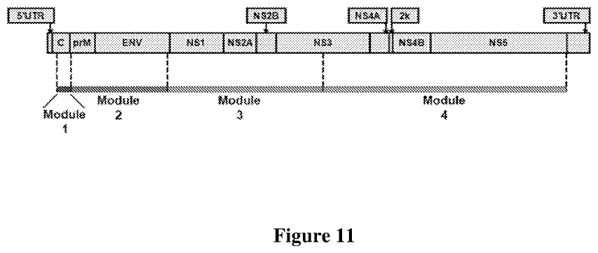

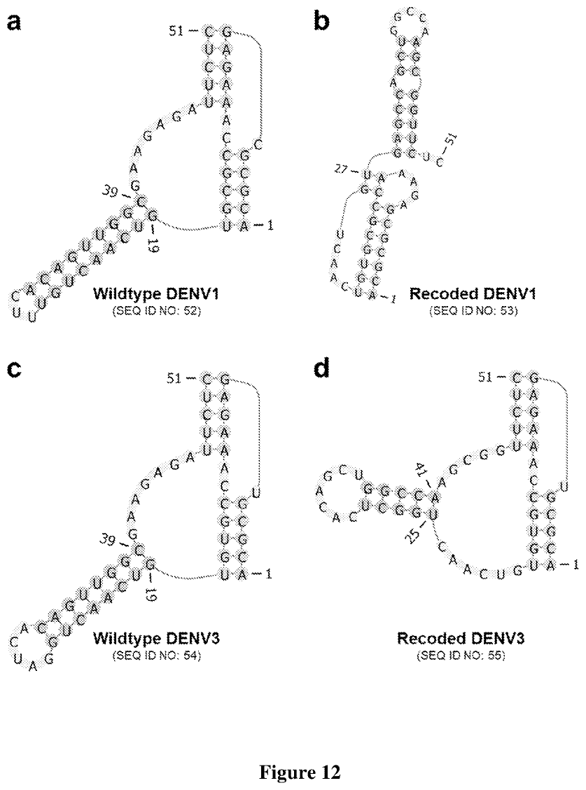

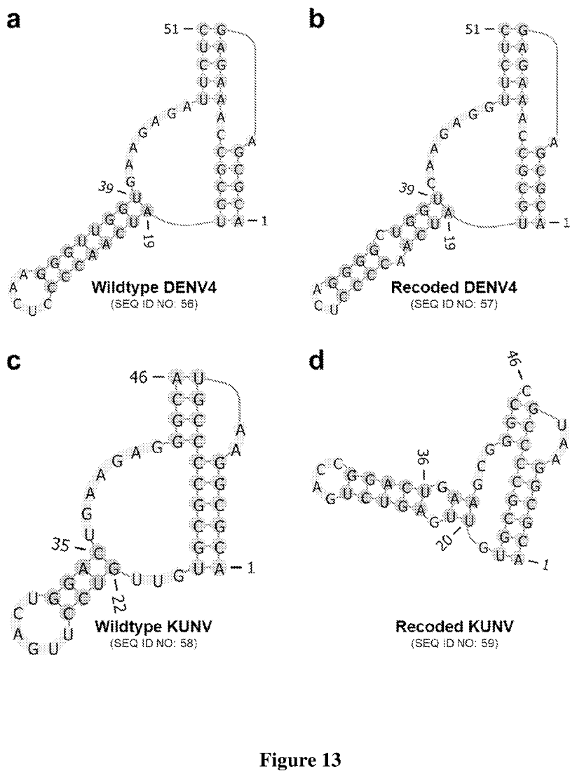

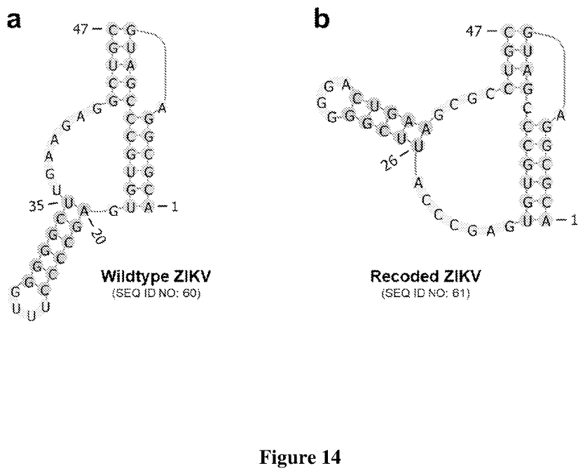

In one embodiment, the synonymous codons are randomly distributed within the portion of the protein coding sequence that comprises or consists of the C coding sequence, or a fragment thereof. For example, the synonymous codons may be randomly distributed within module 1 as defined in FIG. 11.

In one embodiment, the synonymous codons are randomly distributed within the portion of the protein coding sequence that comprises or consists of the prM and E coding sequences, or a fragment thereof. For example, the synonymous codons may be randomly distributed within module 2 as defined in FIG. 11. In one embodiment, the synonymous codons are randomly distributed within the portion of the protein coding sequence that comprises or consists of the E coding sequence, or a fragment thereof.

In one embodiment, the synonymous codons are randomly distributed within the portion of the protein coding sequence that comprises or consists of the NS1, NS2A, NS2B and NS3 coding sequences, or a fragment thereof. For example, the synonymous codons may be randomly distributed within module 3 as defined in FIG. 11. In one embodiment, the synonymous codons are randomly distributed within the portion of the protein coding sequence that comprises or consists of the NS1 coding region, or a fragment thereof.

In one embodiment, the synonymous codons are randomly distributed within the portion of the protein coding sequence that comprises or consists of the NS4A, NS4B and NS5 coding sequences, or a fragment thereof. For example, the synonymous codons may be randomly distributed within module 4 as defined in FIG. 11.

In one embodiment, the synonymous codons are randomly distributed within the portion of the protein coding sequence that comprises or consists of the C, prM and E coding sequences, or a fragment thereof. For example, the synonymous codons may be randomly distributed within modules 1 and 2 as defined in FIG. 11.

In one embodiment, the synonymous codons are randomly distributed within the portion of the protein coding sequence that comprises or consists of the C, prM, E, NS1, NS2A, NS2B and NS3 coding sequences, or a fragment thereof. For example, the synonymous codons may be randomly distributed within modules 1, 2 and 3 of FIG. 11.

In one embodiment, the synonymous codons are randomly distributed within the entire protein coding sequence of the viral genome, i.e., within the single open reading frame (ORF) of the viral genome. For example, the synonymous codons may be randomly distributed within modules 1, 2, 3, and 4 of FIG. 11.

In some embodiments, the method modifies the viral genome to have a protein coding sequence having at least about 70% sequence identity to SEQ ID NO: 4, 6, 8, 12, 14, 18, 20, 24, 26, 30, 32, 36, 38, 42 or 44. The viral genome may have a protein coding sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO: 4, 6, 8, 12, 14, 18, 20, 24, 26, 30, 32, 36, 38, 42 or 44.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U, I) or the identical amino acid residue (e.g., Ala, Arg, Asn, Asp, Cys, Gln, Glu, Gly, His, Ile, Leu, Lys, Met, Phe, Pro, Ser, Thr, Trp, Tyr and Val) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl Acids Res 25:3389. A detailed discussion of sequence analysis can be found in Unit 19.3 of Ausubel et al., “Current Protocols in Molecular Biology”, John Wiley & Sons Inc, 1994-1998, Chapter 15.

In some embodiments, the method further comprises modifying codon 53 of the NS1 gene to code for alanine (A), arginine (R), aspartic acid (D), glutamine (N), lysine (K), methionine (M) or serine (S). In one embodiment, codon 53 of the NS1 gene is modified to TCA or ATG.

The inventors have discovered that the G53A, G53R, G53D, G53N, G53K, G53M and G53S mutations in the flaviviral NS1 gene are broad-spectrum attenuating mutations which reduce viral replication in mosquito-borne flaviviruses. These mutations can further attenuate viral replication on top of attenuation arising from synonymous codon replacement. The inventors have also discovered that specific codons at codon 53 of the NS1 gene, namely the codons TCA (which encodes Ser) and ATG (which encodes Met), are reversion resistant codons, i.e., they have an exceedingly low possibility of reverting to the parent codon (encoding Gly) and can therefore stabilize the respective mutations at that codon position.

In some embodiments, the method modifies the viral genome to have a protein coding sequence having at least about 70% sequence identity to SEQ ID NO: 73, 75, 77, 79, 81, 83 or 85. The viral genome may have a protein coding sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO: 73, 75, 77, 79, 81, 83 or 85.

Disclosed herein is a modified flavivirus obtained according to the method as defined herein.

Also disclosed herein is a modified flavivirus comprising a viral genome comprising a modified virus protein coding sequence having one or more synonymous codons as compared to the protein coding sequence of a parent virus, wherein the modified viral genome is associated with increased protein expression as compared to the viral genome of the parent virus in a mammalian host.

In some embodiments, the modified flavivirus is an attenuated flavivirus. The attenuated flavivirus may exhibit attenuated replication in both a mammalian host (e.g., in humans) and in a non-mammalian host (e.g., in arthropods, preferably in ticks and mosquitoes).

Disclosed herein is an isolated polynucleotide comprising a nucleic acid sequence encoding a modified flavivirus as defined herein. In one embodiment, the polynucleotide is a DNA polynucleotide.

Also disclosed herein is an expression construct comprising a polynucleotide as defined herein. The expression construct may comprise a promoter operably linked to the nucleic acid sequence encoding the modified flavivirus.

Also disclosed herein is a vector comprising a polynucleotide as defined herein.

By “vector” is meant a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

There is also provided herein a immunogenic composition comprising a modified flavivirus as defined herein.

The immunogenic composition may comprise one, two, three, four or more different modified flaviviruses. The immunogenic composition may be a formulation or mixture of modified flaviviruses. For example, the immunogenic composition may comprise a formulation or mixture of four modified Dengue viruses (such as DENV1, DENV2, DENV3 and DENV4). The immunogenic composition may be tetravalent against all 4 DENV serotypes.

An “immunogenic composition” as defined herein may comprise a pharmaceutically acceptable carrier or excipient. A pharmaceutically acceptable carrier or excipient according to the present invention can be any solvent or dispersing medium etc., commonly used in the formulation of pharmaceuticals and immunogenic compositions to enhance stability, sterility and deliverability of the active agent and which does not produce any secondary reaction, for example an allergic reaction, in humans. The excipient is selected on the basis of the pharmaceutical form chosen, the method and the route of administration. Appropriate excipients, and requirements in relation to pharmaceutical formulation, are described in “Remington's Pharmaceutical Sciences” (19th Edition, A. R. Gennaro, Ed., Mack Publishing Co., Easton, Pa. (1995)).

An immunogenic composition of the present invention may optionally contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, human serum albumin, essential amino acids, nonessential amino acids, L-arginine hydrochlorate, saccharose, D-trehalose dehydrate, sorbitol, tris (hydroxymethyl) aminomethane and/or urea. In addition, the immunogenic composition may optionally comprise pharmaceutically acceptable additives including, for example, diluents, binders, stabilizers, and preservatives.

An immunogenic composition of the present invention may comprise one or more adjuvants to enhance the immunogenicity of the live attenuated viruses. Those skilled in the art will be able to select an adjuvant which is appropriate in the context of this invention. An adjuvant may be used in a vaccine composition of the invention comprising a live attenuated virus, as long as said adjuvant does not impact replication.

Suitable adjuvants include an aluminum salt such as aluminum hydroxide gel, aluminum phosphate or alum, but may also be a salt of calcium, magnesium, iron or zinc. Further suitable adjuvants include an insoluble suspension of acylated tyrosine or acylated sugars, cationically or anionically derivatized saccharides, or polyphosphazenes. Alternatively, the adjuvant may be an oil-in-water emulsion adjuvant, as well as combinations of oil-in-water emulsions and other active agents. Other oil emulsion adjuvants have been described, such as water-in-oil emulsions and water-in-oil-in-water emulsions. Examples of such adjuvants include MF59, AF03, AF04, AF05, AF06 and derivatives thereof. The adjuvant may also be a saponin, lipid A or a derivative thereof, an immunostimulatory oligonucleotide, an alkyl glucosamide phosphate, an oil in water emulsion or combinations thereof. Examples of saponins include Quil A and purified fragments thereof such as QS7 and QS21.

As appreciated by skilled persons, an immunogenic composition of the present invention is suitably formulated to be compatible with the intended route of administration. Examples of suitable routes of administration include for instance intramuscular, transcutaneous, subcutaneous, intranasal, oral or intradermal.

The immunogenic compositions of the present disclosure may be administered using conventional hypodermic syringes or safety syringes such as those commercially available from Becton Dickinson Corporation (Franklin Lakes, N.J., USA) or jet injectors. For intradermal administration, conventional hypodermic syringes may be employed using the Mantoux technique or specialized intradermal delivery devices such as the BD Soluvia™ microinjection system (Becton Dickinson Corporation, Franklin Lakes, N.J., USA), may be used.

The volume of an immunogenic composition of the present invention administered will depend on the method of administration. In the case of subcutaneous injections, the volume is generally between 0.1 and 1.0 ml, preferably approximately 0.5 ml.

Disclosed herein is a modified flavivirus, vector or immunogenic composition as defined herein, for use as a medicament or vaccine.

Disclosed herein is a method of modulating an immune response in a subject, the method comprising administering a therapeutically effective amount of a modified flavivirus, vector or immunogenic composition as defined herein to the subject.

The term “modulating an immune response” may comprise inducing an immune response in a subject. This includes stimulating an immune response and/or enhancing a previously existing immune response.

The method may comprise administering a first or prime dose of a modified flavivirus, vector or immunogenic composition, followed by one or more boost doses of the same modified flavivirus, vector or immunogenic composition after a suitable period of time. The first of the one or more boost doses may be administered, for example, between one weeks and ten years after the prime dose, for example, about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about two years, about three years, about four years, about five years, about six years, about seven years, about eight years, about nine years, or about ten years after the prime dose. As a non-limiting example, a first boost dose can be administered about 1 month after the prime dose, and about six months after the first boost dose, a second boost dose can be administered, about 12 months after the second boost dose, a third boost dose can be administered. Additional boost doses can be periodically administered; for example, every 5 years, every 10 years, etc. The timing between the prime and boost doses can vary, for example, depending on the stage of infection or disease (e.g., non-infected, infected, number of days post-infection), and the subject's health. The dosage amount can also vary between the prime and boost dosages. As a non-limiting example, the prime dose can contain fewer copies of the modified flavivirus compared to the boost dose.

The term “subject” as used herein refers to an animal, in particular a mammal and more particularly a primate including a lower primate and even more particularly, a human who can benefit from the present disclosure. A subject regardless of whether a human or non-human animal may be referred to as an individual, subject, animal, patient, host or recipient. For convenience, an “animal” specifically includes livestock animals such as cattle, horses, sheep, pigs, camelids, goats and donkeys, as well as domestic animals, such as dogs and cats. Examples of laboratory test animals include mice, rats, rabbits, guinea pigs and hamsters. Rabbits and rodent animals, such as rats and mice, provide a convenient test system or animal model as do primates and lower primates. In one embodiment, the subject is human.

Disclosed herein is a method of preventing or treating a flaviviral infection in a subject, the method comprising administering to the subject a therapeutically effective amount of a modified flavivirus, vector or immunogenic composition as defined herein.

The method may be used to prevent or treat any infection caused by a flavivirus, or any disease or disorder arising from a flaviviral infection, preferably infections and diseases caused by arthropod-borne flaviviruses, e.g., mosquito- or tick-borne flaviviruses. Non-limiting examples of flaviviral infections include dengue fever, Zika fever, Japanese encephalitis, yellow fever, West Nile fever, Kunjin infection, Murray Valley encephalitis, tick-borne encephalitis, Siberia fever, louping-ill disease, Kyasanur Forest disease, Alkurma hemorrhagic fever, Omsk hemorrhagic fever and Powassan encephalitis.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The methods as disclosed herein may comprise the administration of a “therapeutically effective amount” of an agent (e.g. a vector, modified virus or an immunogenic composition as defined herein) to a subject. As used herein, a “therapeutically effective amount”, when referring to preventing a flaviviral infection, is any non-toxic amount of an agent (e.g., a modified flavivirus, vector or immunogenic composition) that, when administered to a subject prone to flaviviral infection or prone to affliction with a flavivirus-associated disorder, induces in the subject an immune response that protects the subject from becoming infected by the flavivirus or afflicted with the disorder. “Protecting” the subject means either reducing the likelihood of the subject becoming infected with the virus, or lessening the likelihood of the disorder's onset in the subject, by at least two-fold. Most preferably, a therapeutically effective dose induces in the subject an immune response that completely prevents the subject from becoming infected by the flavivirus or prevents the onset of the disorder in the subject entirely.

As used herein, a “therapeutically effective amount”, when referring to treating a flaviviral infection, is any non-toxic amount of an agent that, when administered to a subject afflicted with a flaviviral infection, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Materials and Methods

### Cell Culture and Cell Culture Media

The following cell lines were used in this study: BHK-21 baby hamster kidney cells (ATCC® CCL-10™, USA), Huh-7 human hepatoma cells (kindly provided by Dr. Priscilla Yang, Harvard Medical School, USA), HepG2 human hepatoma cells (ATCC® HB-8065™) Vero E6 African green monkey kidney cells (Vero E6; ATCC CRL-1586™), and C6/36 Aedes albopictus larvae cells (ATCC® CRL-1660™, USA). BHK-21 cells were cultured in Roswell Park Memorial Institute 1640 (RPMI) medium (Sigma-Aldrich) supplemented with 10% foetal calf serum (FCS) and 2 g/L of NaHCO₃. Huh-7, HepG2, and Vero cells were cultured in Dulbecco's Modified Eagle's medium (DMEM) (Sigma-Aldrich) supplemented with 10% FCS and 2 g/L of NaHCO₃. BHK-21, Huh-7, HepG2, and Vero cells were cultured in an incubator at 37° C. with 5% CO2. C6/36 cells were cultured in Leibovitz-15 medium (L-15 medium) (Sigma-Aldrich) in an incubator at 28° C. without additional CO₂.

### Viruses

For this study we used DENV2 strain 16681 (GenBank accession no. NC_001474.2) and ZIKV strain PRVABC59 (GenBank accession no. KU501215.1). Wildtype and recoded viruses were rescued from their respective infectious clones by DNA-launch in BHK-21 cells (see below).

### Virus Titration

Virus titration was performed using plaque assay or fluorescent focus formation assay in BHK-21 cells. For plaque assay, BHK-21 cells were seeded one day before virus inoculation in a 24-well plate at a density of 5×10⁴ cells per well. To prepare for virus inoculation, virus stocks were serially diluted 10-fold in RPMI medium supplemented with 2% FCS and 2 g/L of NaHCO₃. Next, the cell culture medium was removed from the BHK-21 cells, and then each well was inoculated with 100 l of the serially diluted virus stock. The cells and virus were then incubated in an incubator at 37° C. with 5% CO₂ for 1 hour. After incubation, the supernatant containing the virus was removed from the wells, and then the cells were washed twice with 1 ml of PBS per well. After washing, the cells were overlaid with RPMI medium supplemented with 2% FCS, 2 g/L of NaHCO₃, and carboxymethyl cellulose (CMC). The inoculated cells were then incubated in an incubator at 37° C. with 5% CO₂ for 8 days. For DENV2, the CMC concentration was 0.8%, and the cells were incubated for 8 days after inoculation. For ZIKV, the CMC concentration was 1%, and the cells were incubated for 6 days after inoculation. After incubation, the cells and plaques were fixed and stained with a solution containing 10% paraformaldehyde and 1% crystal violet.

For fluorescent focus formation assay, BHK-21 cells were seeded one day before virus inoculation in a 96-well plate at a density of 1.2×10⁴ cells per well. For inoculation, the cell culture medium was removed from the cells, and then each well was inoculated with 40 l of neat virus stock. The cells and virus were then incubated in an incubator at 37° C. with 5% CO₂ for 1 hour. After incubation, the supernatant containing the virus was removed from the wells, and then the cells were washed twice with PBS. After washing, the cells were overlaid with RPMI medium supplemented with 2% FCS and 2 g/L of NaHCO₃. The inoculated cells were then incubated in an incubator at 37° C. with 5% CO₂ for 2 days. After incubation, the cells were fixed using a 4% PFA solution and their nuclei were stained with DAPI. After fixation, the cells were analysed by fluorescent microscopy using an automated Operetta High content imager platform (PerkinElmer). The fluorescent microscopy images were then analysed using the Cell Profiler software to determine the ratio of GFP positive cells to total nuclei count and this ratio was used to calculate the virus concentration in terms of focus forming units per ml (FFU/ml).

### Virus Culture and Viral Growth Kinetics

The virus culture media that was used for virus culture and growth kinetics was the same as the cell culture media of the respective cell line, except that the concentration of FCS was reduced from 10% to 2%.

Viral growth kinetics was performed in BHK-21, Huh-7, HepG2, Vero, and C6/36 cells. The multiplicity of infection (MOI) for experiments was determined by the virus stock with the lowest titre. The cells were seeded one day before virus inoculation in a 24-well plate. Seeding densities per well are as follows: 6×10⁴ cells for BHK-21, 8×10⁴ cells for Huh-7, 1.8×10⁵ cells for HepG2, and 2.5×10⁵ cells for C6/36. To prepare for virus inoculation, the virus stocks were diluted to the appropriate concentration in the virus culture medium of the cell line that was to be infected. Next, the cell culture medium was removed from the 24-well plates, and then each well was inoculated with 200 l of the diluted virus stock. The cells and virus were then incubated in an incubator at 37° C. with 5% CO₂ for 1 hour. After incubation, the supernatant containing the virus was removed from the wells, and then the cells were washed twice with 1 ml of PBS per well. After washing, 1 ml of the appropriate virus culture medium was added to each well. The inoculated human and mammalian cells were then incubated in an incubator at 37° C. with 5% CO₂, while the inoculated C6/36 cells were incubated in an incubator at 28° C. The virus supernatant was harvested once per day after infection until all or almost all of the cells that were infected with wildtype virus developed cytopathic effects.

### Cell Viability Assay

To prepare for cell viability assay, Huh-7 cells were seeded one day before virus inoculation in 96-well plates (Corning) at a density of 1.3×10⁴ cells per well. Virus inoculation was performed as described above at an MOI of 0.02. After virus inoculation, the virus inoculation supernatant was replaced with 100 μL of virus culture medium. Cell viability assay was performed at once per day after infection using alamarBlue™ Cell Viability Reagent (Thermo Fisher). The alamarBlue reagent was first diluted 10-fold using virus culture medium. Next, the virus culture supernatant was first discarded, after which 100 μL of the diluted alamarBlue reagent was added to each well. The plates were then wrapped in aluminium foil and incubated for two to three hours in an incubator at 37° C. with 5% CO₂. The plates were then read using the Infinite™ 200 series microplate reader (Tecan) at emission and excitation wavelengths of 600 nm and 570 nm respectively. Cell viability was normalised to mock infected Huh-7 cells.

### Construction of Recoded Infectious Clones

The infectious clones described in this study were derived from our existing DENV2 and ZIKV infectious clones. These include the previously reported GFP reporter dengue virus 2 (DENV2-GFP), Firefly luciferase (FLuc) translation reporter, and DENV2 subgenomic replicon.

The details of the regions targeted for recoding are detailed in Table 2. The sequence of DENV2-rcCap-NS1, the most extensively recoded clone, is also available on Genbank (accession number OP909734). The recoded DENV2 cDNA sequences were synthesised as short, slightly overlapping fragments of a few hundred base pairs in length. The recoded ZIKV cDNA sequences were synthesised as overlapping fragments of a few thousand base pairs in length. The stability of recoded ZIKV infectious clones allowed their TRE-minCMV promoter to be replaced with a conventional CMV promoter (cloned from pcDNA3.1). The recoded sequences were assembled with each other or wildtype sequences using fusion PCR (Q5 Hot Start High-Fidelity 2× Master Mix, NEB). The assembled sequences were cloned into infectious clone plasmids using conventional molecular cloning techniques: DNA was digested with restriction enzymes (NEB) and ligated using T4 ligase (NEB). Infectious clone plasmids were propagated in Stbl3 E. coli competent cells (Thermo Fisher) that were cultured in LB broth supplemented with 35 g/ml of kanamycin (GoldBio). The infectious clone plasmid sequences were verified by Sanger sequencing (performed by 1st BASE, Axil Scientific).

The reporter DENV2-GFP encodes a recombinant C75-EGFP-P2A-UBB-smC75 cassette, whereby the first 75 nucleotides of the capsid coding region are duplicated (C75 and smC75). The EGFP gene is cloned between these two duplicate regions. The upstream C75 sequence preserves the wildtype sequence and position of two critical RNA elements, the capsid coding region hairpin element (cHP) and the 5′ cyclisation sequence (5′CS). The downstream smC75 sequence is codon optimised to abrogate the duplicated cHP and (5′ CS) with multiple silent mutations (essentially recoding them). For the recoded DENV2 and ZIKV clones that do not encode EGFP, the first 75 nucleotides of the capsid coding region are not codon optimised to avoid targeting the critical 5′CS and cHP elements as it is suspected that there would be strong selection pressure for reversion mutations if we they were not codon-optimised. For the ZIKV clones, the 3′ boundaries of the recoded regions corresponded to BsiWI and AflII restriction sites found in the NS3 and NS5 coding regions respectively.

It is noted that in the design of the DENV2-rcCap-Env clone, the first 75 nucleotides of the Capsid coding region and last 75 nucleotides of the Envelope coding region are deliberately left unmodified, and only the codons that lie in between these two regions are recoded. This design is derived from prior flavivirus subgenomic replicons, which were constructed by deleting this same stretch of nucleotides lying between the first 75 nucleotides of the capsid coding region and the last 75 nucleotides of the envelope coding region. These subgenomic replicons lack the expression of structural proteins but retain the ability to undergo RNA replication, indicating that the RNA elements in the deleted region are not strictly essential for RNA replication.

Virus Rescue from Infectious Clones

Virus rescue from infectious clone plasmids was performed by DNA launch in BHK-21 cells. Recoded Zika virus clones had a conventional CMV promoter and could be DNA launched by transfecting the infectious clones by themselves. All other infectious clones were DNA launched by co-transfecting the infectious clone plasmid with the pTet-Off Advanced accessory plasmid (400 ng of viral plasmid for every 100 ng of accessory plasmid).

BHK-21 cells were seeded one day before transfection in a 6-well plate at a density of 2.4×10⁵ cells per well. The transfection was performed using jetPRIME (Polyplus transfection) according to the manufacturer's instructions; each well was transfected with a total of 2,000 ng of DNA. Five hours after transfection, the cell culture medium was changed to virus culture medium. The transfected cells were then incubated in an incubator at 37° C. with 5% CO₂. The virus supernatant was harvested when the cells started to show CPE: the virus supernatant was filtered using a Sartorius syringe driven 0.22 micron PES filter and then aliquoted before being stored at −80° C. We consider this to be the passage 1 virus stock.

The wildtype and recoded DENV2 passage 1 stocks were propagated once in BHK-21 cells to produce a passage 2 working stock. The titres of the wildtype and recoded ZIKV passage 1 stocks were high enough for cell culture experiments; no further passaging was required.

### Fluorescent Microscopy

Live imaging of infected cells was performed using GFP reporter DENV2 (DENV2-GFP). The wildtype and recoded DENV2-GFP clones were DNA-launched in BHK-21 cells as described above. The cells were imaged live using fluorescent microscopy was performed on an EVO FL digital inverted fluorescence microscope (Thermo Fisher).

### Translation Reporter Luciferase Assay and Subgenomic Replicon RNA Replication Assay

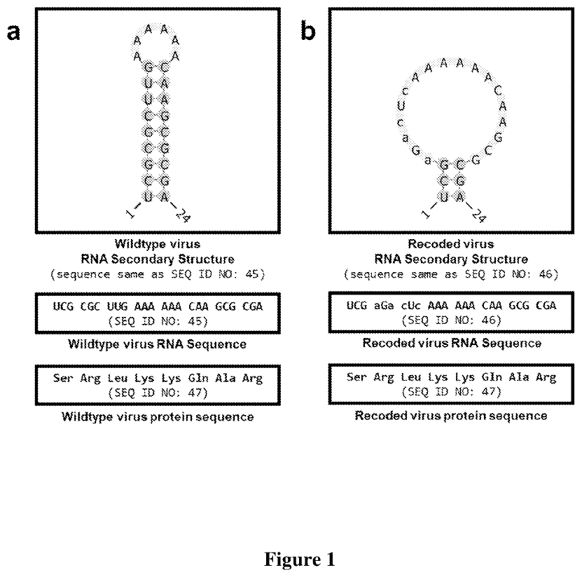

The FLuc translation reporter was used to study the impact of recoding on viral protein translation efficiency. The translation reporter construct can undergo viral protein expression, but is unable to undergo RNA replication due to a deletion of the GDD catalytic triad RNA-dependent RNA polymerase domain of the NS5 protein (FIG. 1b). The translation reporter construct expresses firefly luciferase as a reporter of viral protein expression. The firefly luciferase assay is the same as previously described.

The subgenomic replicon was used to study the impact of recoding on viral RNA replication. The subgenomic replicon retains the ability to undergo viral protein expression and RNA replication but is limited to a single round of infection because it contains a partial deletion of the capsid protein. The qRT-PCR assay that was used to measure viral RNA replication is the same as previously described.

### Viral RNA Extraction and Fragmentation for Next Generation Sequencing

For each sample, viral RNA was extracted from 200 μL of viral supernatant. First, 600 μL of lysis buffer (Invitrogen) was added to each sample. The mixture was vortexed and left to incubate on ice for 10 minutes.

After incubation, 800 μL of acid phenol:chloroform (Ambion: 5:1, pH 4.5) was added. The mixture was vortexed again and then centrifuged for 5 minutes at 4° C. to separate the phenol-chloroform phase, the interphase, and the aqueous phase. The aqueous phase was transferred to a new tube. An equal volume of 100% isopropanol and 2 μL of Pellet Paint® Co-Precipitant (Novogene) was added to the aqueous phase and the mixture was incubated at room temperature for 5 minutes and then centrifuged for 10 minutes to pellet the RNA. Next, the supernatant was removed, leaving the RNA pellet. The RNA pellet was then washed three times with 70% ethanol, followed by another three washes with 100% ethanol. The final ethanol wash was then removed and the RNA pellet was allowed to air dry. Finally, the RNA was resuspended in 30 μL of nuclease-free water.

Viral RNA fragmentation was performed using the NEBNext® Magnesium RNA Fragmentation Module (NEB), according to the manufacturer's instructions. Viral RNA was fragmented in 2 μL of NEBNext® Magnesium RNA Fragmentation Buffer at 94° C. for two minutes, after which the reaction was stopped by adding 2 μL of NEBNext® Fragmentation Stop solution. To purify the fragmented RNA, 60 μL of 100% ethanol, 2 μL of 3M sodium acetate, and 2 μL of Pellet Paint® was added to each sample. The mixture was incubated at room temperature for 5 minutes, after which the mixture was centrifuged at 4° C. for 10 minutes to pellet the RNA. The supernatant was then removed and the RNA pellet was washed once with 70% ethanol and then washed one more time with 100% ethanol. The RNA pellet was allowed to air dry before being resuspended in 15 μL of nuclease-free water.

Viral cDNA Synthesis and Library Prep for NGS

Double stranded cDNA was synthesised from the fragmented viral RNA samples using the Maxima H Minus Double-Stranded cDNA Synthesis Kit (Thermo Fisher), according to the manufacturer's instructions. The first strand cDNA synthesis was primed using random hexamer primers. After double stranded cDNA synthesis, residual RNA was removed by adding 10 μL of RNase I to the 100 μL double stranded cDNA mixture. The RNase reaction was incubated at room temperature for five minutes, after which the double stranded cDNA was purified using the NucleoSpin® Gel and PCR Clean-up kit (Macherey-Nagel). Finally, the purified cDNA was eluted in 55 μL of Tris-HCl.

Library preparation was performed using the KAPA HyperPrep Kit (Roche Sequencing Solutions; USA) according to the SeqCap® EZ HyperCap Workflow. End repair and A-tailing was performed on the samples for adapter ligation. The ligated products were cleaned up using Agencourt AMPure XP beads. The samples were then amplified using ligation-mediated polymerase chain reaction (LM-PCR), after which the samples were purified using Agencourt AMPure XP beads. Finally, Agencourt AMPure XP beads were used for size selection for fragments that were 250 bp to 450 bp in size.

The library was quantified using the Qubit dsDNA High Sensitivity Assay Kit and Qubit fluorometer (Thermo Fisher) while the library quality was verified using the Agilent 2100 Bioanalyzer.

### Next Generation Sequencing

The samples were sequenced by GENEWIZ using NovaSeq 6000 (Illumina). Reads were filtered using Genome Detective (v1.132) and alignment analysis was conducted using Geneious Prime (v2021.0.3) (Biomatters).

### RNA Pseudoknot Structure Prediction and Visualisation

RNA pseudoknot structures were modelled using pKiss, using the thermodynamic parameters that were previously published. The predicted pseudoknot structures were visualised using PseudoViewer.

### Neurovirulence Studies in Suckling Mice

The in vivo attenuation of DENV2 was characterised in a suckling mouse model of neurovirulence. Newborn outbred white ICR mice from InVivos, Singapore, were inoculated by intracranial injection within 24 hours of birth with DENV2 that had been diluted to a concentration of 10⁵ PFU/ml.

### SHAPE-MaP Structure Probing of DENV2 Viruses in BHK-2 Cells

BHK-21 cells were infected with DENV2 (WT and mutant) at a multiplicity of infection (MOI)=0.01 for 1 h at 37° C. Following 1 h infection, virus inoculum was removed and replaced with DMEM-5% FBS. Flasks were incubated for 48 h at 37° C., 5% CO₂. At 48 hours post infection, cells were washed once with PBS and trypsin was added to detach the cells from the flask. The cells were collected and centrifuged at 300×g for 5 min. The pellet was resuspended in PBS and the cells were then separated into three reactions: (1) added 1:20 volume of 1 M NAI (03-310, Merck) and incubated for 15 min at 37° C. for structure probing; (2) added 1:20 volume of dimethyl sulfoxide (DMSO) and incubated for 15 min at 37° C., as negative control; and (3) set aside a third portion of the infected cells without any treatment, for the denaturing control in the downstream library preparation process. One set of uninfected BHK-21 cells were treated as negative control. The total RNA was extracted from the cells using Qiagen RNeasy Mini Kit according to the manufacturer's instructions. Library preparation was performed following the SHAPE-MaP protocol to generate cDNA libraries compatible for Illumina sequencing.

### In Silico RNA Secondary Structure Prediction and Analysis

SHAPE data was obtained using Shapemapper 2.15 independently for 2 technical replicates of the Wildtype and recoded strains respectively. Minimum read depth was set to 1000. Reactivity data at this depth was obtained for 98.8% of Wildtype and 99.2% of recoded RNA positions. Wildtype and recoded genomic RNA sequences were aligned using MAFFT v7.481, yielding a gapless alignment. Differences in SHAPE were assessed using ΔSHAPE. Comparison of SHAPE data confirmed that significant structural changes are largely confined to the recoded region spanning bases 1-2421 in both variants. Therefore, structure modelling was confined to this section. The Superfold pipeline was used with RNAstructure v6.3 as a backend for structure prediction with default parameters incorporating SHAPE data as a constraint. Subsequent analysis shows good agreement between SHAPE data and the resulting RNA structure models, indicating that the models are plausible in light of the experimental evidence. Structures were visualized using VARNA 3.93 and a custom script to map SHAPE reactivity data onto the resulting figures.

### Neurovirulence Studies in Suckling Mice

The in vivo attenuation of DENV2 was characterised in a suckling mouse model of neurovirulence. Newborn outbred white ICR mice from InVivos, Singapore, were inoculated with virus via intracranial injection within 24 hours of birth.

Mosquito challenge studies Mosquito challenge studies was performed by feeding an infectious blood meal to Aedes albopictus mosquitoes. The Aedes albopictus (NEA-EHI strain) colony used for this study is a local Singapore strain that was obtained from the Environmental Health Institute, Singapore. The mosquito colony was maintained in the insectary of Temasek Life Sciences Laboratory. The blood meal was prepared from rabbit blood that was freshly drawn on the day of oral infection. The blood was centrifuged at 2,500 rpm and 4° C. for 10 min to separate there blood cells from the serum. The serum was then heat-inactivated at 55° C. for 1 hour.

The blood cells were washed three times with PBS. The heat-inactivated serum and washed blood cells were then mixed together and supplemented with 1 mM of ATP. The treated blood was then mixed with diluted virus stock at a 1:1 ratio, to obtain an infectious titre of 2.5×10⁷ PFU/ml.

Oral infection was performed using female Aedes albopictus mosquitoes at day 5 after emergence. The mosquitoes were sugar starved overnight prior to the oral infection. The mosquitoes were fed the infectious blood meal using the Hemotek system (PS5, Hemotek Ltd England). After oral infection, partially engorged or unfed mosquitoes were removed from the cage. The remaining engorged females were kept for 11 days at 28° C. with 80% humiditiy and a photoperiod of 12:12 hours (light:dark) with 10% sucrose solution provided ad libitum. After an 11 day incubation period, the mosquitoes were collected to determine their infection status and viral load. Individual mosquitoes were homogenised in 100 l of PBS after which their infection status and viral load was determined using plaque assay.

### Statistical Analysis

Statistical analysis and graph plotting was performed using GraphPad Prism 9. Viral growth kinetic titres, firefly luciferase assay, qRT-PCR assay, and viral plaque sizes were compared using one-way ANOVA, with Tukey's multiple comparisons post hoc test. Data for Viral growth kinetic titres, firefly luciferase assay, and qRT-PCR assay are representative of the mean of three replicates. Bonferroni correction was used for when performing multiple comparisons for mouse survival. Plaque sizes were measured in ImageJ using the ViralPlaque Fiji macro. Figures for plaque sizes indicate the mean and standard deviation of at least thirty plaques and are representative of at least two biological replicates. Non-parametric unpaired T-test was used for mosquito challenge.

### Example 1

### Virus Genome Recoding

There have been several studies on recoding mosquito-borne flaviviruses: DENV2, ZIKV, and YFV. The recoding was performed by de-optimising the virus genome by targeting factors such as codon pair bias, or dinucleotides, or even TCG trinucleotides and the results generally showed attenuation in mammalian cells or in mammalian animal models.

However, the de-optimisation was usually performed by optimising the virus for an arthropod or mosquito host at the expense of a mammalian or human host. While the results generally showed attenuation in mammalian cells or in mammalian animal models, the results were inconsistent. In one study, de-optimised DENV2 showed poor replication in LLC-MK2 monkey cells, but replicated normally in BHK-21 cells. Furthermore, the main disadvantage of this recoding approach is that it does not result in attenuation in mosquito cells or mosquitos. These studies found that mammalian de-optimised DENV2 and ZIKV could replicate normally in C6/36 cells, while the mammalian de-optimised DENV2 remained transmissible in Aedes mosquitoes. This is a major shortcoming because live attenuated flavivirus vaccines must lack transmissibility in their mosquito vectors.

### Approach

The flavivirus genome contains many functional RNA elements that are essential for efficient virus replication. These functional RNA elements may take the form of pseudoknots, RNA secondary structures, or long-range RNA interactions that help to regulate viral protein expression or RNA replication. These RNA elements are spread across the entire flavivirus genome, in the 5′ and 3′ untranslated regions (5′UTR and 3′UTR), as well as in the protein coding region. For example, the capsid coding region of the mosquito-borne flaviviruses contains conserved RNA elements such as the capsid coding region hairpin element (cHP), the 5′ cyclisation sequence (5′ CS), and the downstream of 5′ cyclization sequence pseudoknot (DCS-PK).

One of the major functions of these RNA elements is regulating viral RNA replication. RNA elements in the 5′UTR, 3′UTR, and protein coding region play a role in the cyclisation of the viral RNA genome, a process which is required for the virus genome to transition from protein translation to RNA replication. Prior studies have shown that silent mutations that target the cHP, 5′ CS, or DCS-PK elements can inhibit genome cyclisation, which in turn reduces viral RNA replication efficiency.

The protein coding region of the flavivirus genome is predicted to possess many more RNA elements. The function of many of these predicted RNA elements have yet to be experimentally verified. Nonetheless, early studies have shown that abrogating the sequence and function of some of these predicted RNA elements with silent mutations can have a small but measurable impact on viral replication. Recoding of the flavivirus genome with silent mutations can potentially abrogate the sequence and function of many of these RNA elements (FIG. 1).

The individual deleterious effects of abrogating these minor RNA elements would not be as significant as targeting the more critical cHP or 5′ CS elements. However, it would ensure the genetic stability of the recoded virus because there would be very little selection pressure for an individual mutation to revert. If degree of recoding were extensive enough, the cumulative effect of these mutations would be sufficient to reduce viral RNA replication efficiency to such an extent that the virus would be attenuated. The mechanism of this attenuation targets something inherent to flaviviruses; it will function consistently regardless of cell type, pattern recognition receptor (PRR) profile, or animal species.

Large regions of the flavivirus genome may be codon optimized in favour of human expression. While codon optimisation will allow recoded viruses to achieve higher protein translation efficiency in human cells, this may be balanced out by a reduction in RNA replication efficiency. An additional benefit is that the recoded virus will have both reduced protein translation efficiency and reduced RNA replication efficiency in mosquito cells, which will help prevent transmission by mosquitoes.

This recoding approach is quick and simple because it does not require mapping the RNA structures of any given flavivirus. The codon optimisation process introduces silent mutations that are evenly spaced out across approximately 58% to 61% of codons (Table 2). This may be enough to disrupt RNA elements lying within any region targeted for recoding.

It is also worth mentioning that virus genome recoding does not affect the encoded viral protein sequences. This means that the viral antigen sequences remain unaffected, and viral protein function remains unchanged, so any differences in viral phenotype would be due to differences at the functional RNA level. It also means that this recoding approach can be combined with other attenuation methods, such as deleting a portion of the 3′UTR, to rescue vaccine strains that had failed due to insufficient attenuation.

### Example 2

### Initial Characterisation of Recoded DENV2-GFP Clones

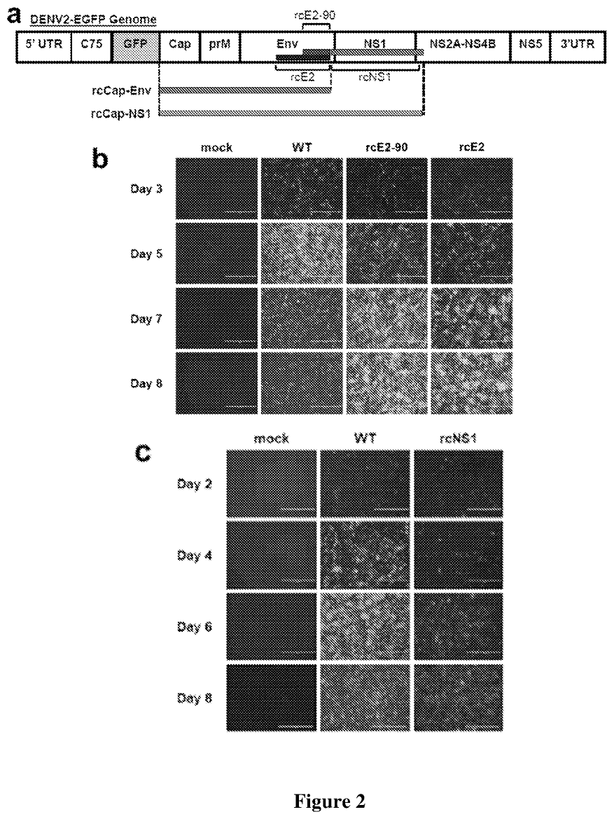

DENV2-GFP is a recombinant dengue type 2 serotype virus that expresses EGFP as a reporter protein. Codon optimised sequences were assembled and cloned into the DENV2-GFP infectious clone. The rcE2-90 and rcE2 clones were constructed (FIG. 2a & Table 2), with the respective recoded regions corresponding to codons 381 to 470 and codons 201 to 470 of the envelope protein (Env) coding region. The rcNS1 clone was also constructed, with the recoded region corresponding to the last 25 codons of the Env coding region, all of the NS1 coding region, and the first 25 codons of the NS2A coding region (402 codons total) (FIG. 2a & Table 2).

The effects of genome recoding on DENV2-GFP replication was investigated by DNA-launching wildtype (non-recoded) and recoded DENV2-GFP clones in BHK-21 cells. After DNA-launch, the cells were imaged using fluorescent microscopy, and EGFP expression was used as a marker of virus infection (FIG. 2b & 2c). It was found that the rcE2-90, rcE2, and rcNS1 clones showed slower cell to cell spread compared to wildtype (non-recoded) DENV2-CFP, as indicated by the slower spread of the GFP signal (FIG. 2b & 2c). This indicates that virus genome recoding by codon optimisation can reduce DENV2 replication efficiency.

Recoded Virus Clones have Higher Protein Expression Efficiency but Lower RNA Replication Efficiency

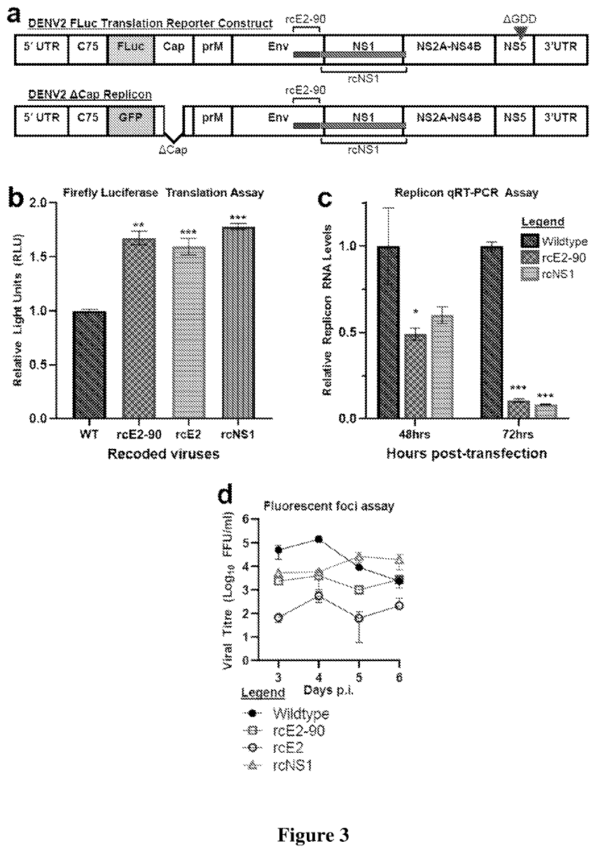

Next, the mechanism of action of DENV2 genome recoding was investigated. To study viral protein expression, a translation reporter construct was used that is unable to undergo RNA replication due to a deletion of the GDD catalytic triad RNA-dependent RNA polymerase domain of the NS5 protein (FIG. 3a). The translation reporter can still express the viral polyprotein, but it never enters the exponential protein expression and RNA replication phase. The translation reporter construct expresses firefly luciferase as a reporter of viral protein expression.

To study viral RNA replication, a subgenomic replicon that can express viral proteins and undergo RNA replication (FIG. 3a) was used. However, the subgenomic replicon does not undergo viral particle packaging because it contains a partial deletion of the capsid protein. Therefore, the replicon is limited to a single round of intracellular replication, and cannot spread from cell to cell.

The recoded rcNS1 and rcE2-90 mutations were cloned into the translation reporter construct (FIG. 3a) and then DNA-launched into BHK-21 cells. After DNA-launch, firefly luciferase activity was measured to determine viral protein translation efficiency. It was found that the recoded reNS1, rcE2-90, and rcE2 constructs had higher firefly luciferase activity compared to the wildtype control (FIG. 3b). This indicates that the recoded constructs have higher viral protein translation efficiency relative to the wildtype.

Next, the rcE2-90 and rcNS1 mutations were cloned into the subgenomic replicon and then DNA-launched into BHK-21 cells. After DNA-launch, replicon RNA levels were measured to determine viral RNA replication efficiency (FIG. 3c). It was found that the recoded replicons had a lower RNA replication efficiency, as the rcE2-90 and rcNS1 replicons had an almost 10-fold lower RNA level compared to the wildtype replicon by at day 3 post transfection.

Thus, codon optimising the DENV2 genome for human expression appears to enhance viral protein translation efficiency in mammalian cells, but this appears to be balanced out by a loss of viral RNA replication efficiency. This is consistent with the abrogation of functional RNA elements that regulate the transition of the DENV2 RNA genome from the linear protein translation state to the competing circularised RNA replication state. The overall effect is a loss of viral replication efficiency.

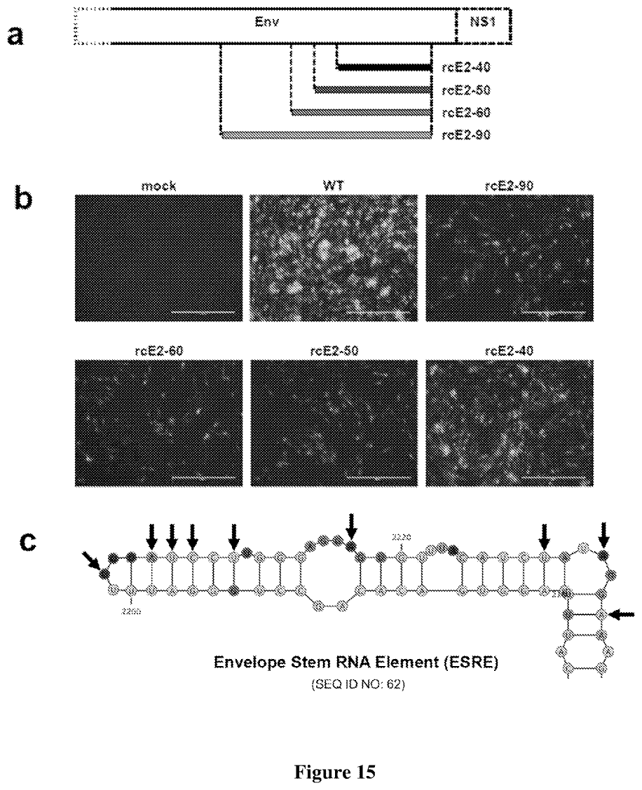

The envelope stem coding region contains a putative RNA element Three additional recoded clones were constructed: rcE2-60, rcE2-50, and rcE2-40, with the respective recoded regions narrowed down to 60, 50, and 40 codons respectively (FIG. S2a & Table 2). The rcE2-60 and rcE2-50 recoded clones retained the reduced replication efficiency of the rcE2-90 clone, while the rcE2-40 clone replicated faster (FIG. S2b). The rcE2-50 clone differs from the rcE2-40 clone by a region corresponding to nucleotides 2197 to 2226 of the DENV2 genome and codons 421 to 430 of the envelope protein coding region. Codons 421 to 430 encode for the envelope protein stem region. In wildtype DENV2, nucleotides 2197 to 2226 are predicted to be part of a RNA hairpin structure (FIG. 15c & 10b), and the recoding mutations are predicted to disrupt this RNA hairpin. Therefore, this RNA hairpin may be a RNA element that contributes to efficient DENV2 replication. This putative RNA element is named the envelope stem RNA element (ESRE) in this disclosure.

Degree of Recoding is Correlated with Slower DENV2-GFP Replication

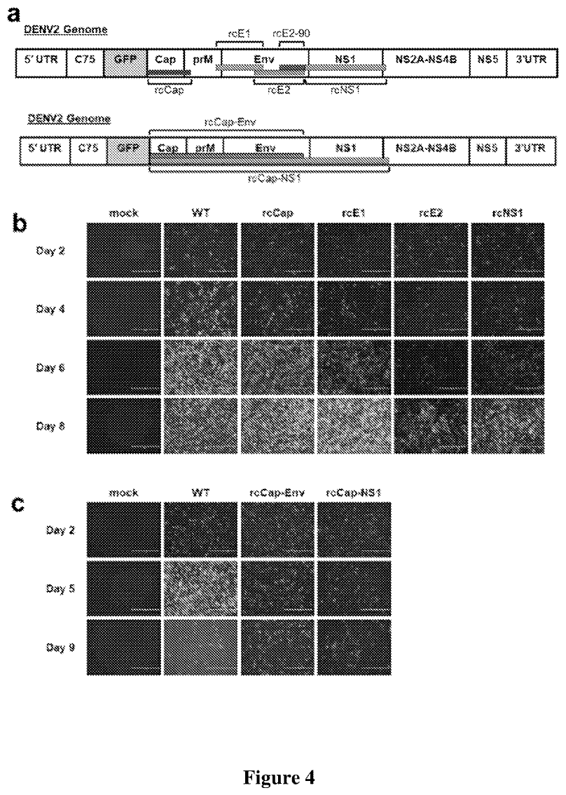

The effects of codon optimising in other regions of the DENV2 genome were investigated (FIG. 4a & Table 2) by constructing rcCap, with the recoded region starting with the entire capsid coding region and ending with the first 25 codons of the premembrane (prM) coding region, andrcE1, with the recoded region starting with the last 25 codons of the prM coding region followed by the first 810 nucleotides (270 codons) of the Env coding region.

It was found that the recoded DENV2-GFP clones, rcCap, rcE1, rcE2, and rcNS1, all had slower cell to cell spread compared to wildtype (non-recoded) DENV2-GFP, as indicated by the slower spread of the GFP signal (FIG. 4b). This confirms that virus genome recoding by codon optimisation can reduce DENV2 replication efficiency.

Next, it was investigated whether increasing the degree of genome recoding would lead to an increasing degree of attenuation (FIG. 4a) The recoded sequences were combined to construct rcCap-Env and rcCap-NS1 (FIG. 4a & Table 2). It was observed that the rcCap-Env and rcCap-NS1 clones replicated so slowly that there was no significant spread of the GFP reporter signal. This indicates that increasing the degree of genome recoding resulted in a greater reduction in virus replication efficiency.

Recoded DENV2 Clones have Reduced Replication Efficiency

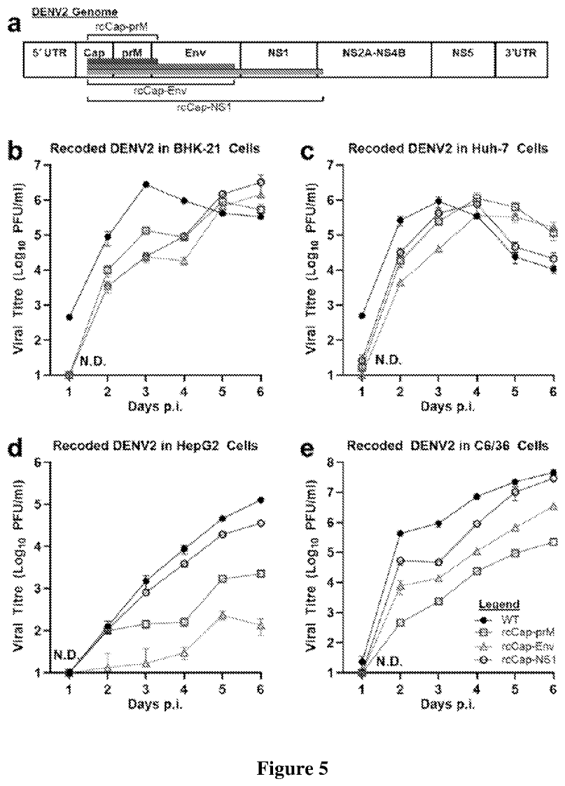

To avoid potential experimental artifacts arising from the use of the GFP reporter virus, three recoded DENV2 clones were constructed that were based on a wildtype DENV2-16681 backbone that does not carry any trans-genes. These clones were named rcCap-prM, rcCap-Env, and rcCap-NS1 to correspond to the regions in the genome that were targeted for recoding (FIG. 5a & Table 2). To construct these clones, the transgenic EGFP-P2A-UBB-smC75 cassette was deleted from the DENV2-GFP clones without modifying the 5′CS or cHP RNA elements.

The growth kinetics of the wildtype and recoded DENV2 clones were compared in BHK-21 hamster kidney cells, Huh-7 human hepatocarcinoma cells, HepG2 hepatoma cells, and C6/36 Aedes mosquito cells. The cells were infected at an MOI of 0.02 and the viral titres were measured using plaque assay. The growth kinetics with DENV2 confirms that our recoded clones have reduced replication efficiency (FIG. 5). When compared to the wildtype virus, the titres for the recoded DENV2 clones peaked at later timepoints in BHK-21, Huh-7, and HepG2 cells, even though the recoded clones had been codon optimised for human cells. In HepG2 cells, the peak titres of the recoded DENV2 clones were also lower when compared to wildtype DENV2 (FIG. 5d). In C6/36 cells, the titres of the recoded DENV2 clones were lower than the wildtype virus at all timepoints (FIG. 5e). This reduced replication efficiency in mosquito cells is favourable because it is a key marker of attenuation that is correlated with reduced transmissibility by mosquitoes. The rcCap-Env clone had the lowest replication efficiency in BHK-21, Huh-7, and HepG2 cells, while the rcCap-prM clone had the lowest replication efficiency in C6/36 cells.

It is worth pointing out that genome recoding leaves the encoded viral protein sequence intact, which means viral protein function remains unchanged. Therefore, any differences in replication efficiency will be due to changes at the functional RNA level. These results confirm that virus genome recoding by codon optimization can reduce virus replication efficiency regardless of cell type or animal species.

Recoded ZIKV Clones have Reduced Replication Efficiency

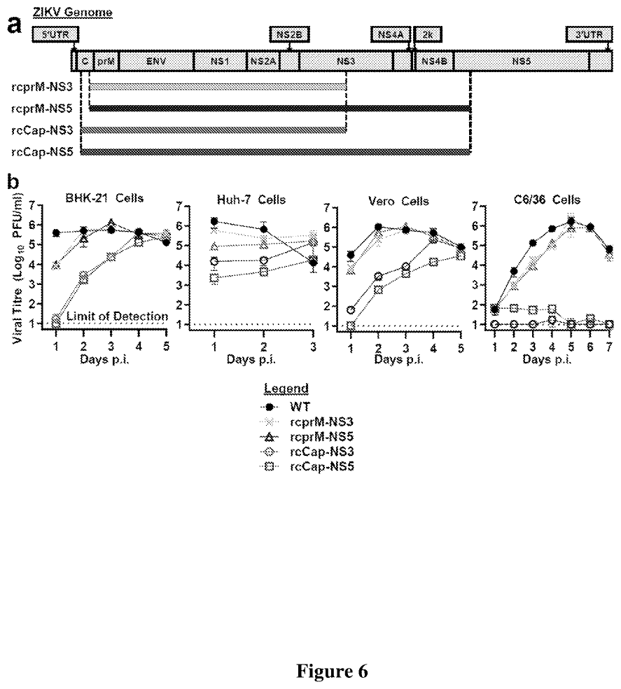

To investigate if the recoding approach also works on another flavivirus, recoded ZIKV clones, rcprM-NS3, rcprM-NS5, rcCap-NS3, and rcCap-NS5, were constructed, with the names roughly corresponding to the regions in the genome that were targeted for recoding (FIG. 6a and Table 2).

Viral growth kinetics of wildtype and recoded ZIKV clones were investigated in Huh-7, Vero E6, BHK-21, and C6/36 cells. Vero E6 was used in place of HepG2 because ZIKV does not replicate as well in HepG2 cells. The cells were infected at an MOI of 0.01 and viral titres were measured using plaque assay. When compared to the wildtype virus, the recoded ZIKV clones demonstrated reduced replication efficiency in mammalian, human, and mosquito cells. In general, recoded ZIKV clones replicated to lower peak virus titres, and their viral titres peaked at later timepoints (FIG. 6)

Taking the ZIKV-rcprM-NS3 clone as the baseline, recoding of the prM to NS3 coding regions in the ZIKV-rcprM-NS3 clone conferred a mild reduction in virus replication efficiency. Thereafter, increasing the degree of recoding resulted in further reductions in virus replication efficiency. The degree of reduction depended on which region was the target of further recoding: the NS3 to NS5 coding region had the smallest effect, the capsid coding region by itself had a greater effect, and targeting both the capsid and NS3 to NS5 coding regions had the greatest effect (FIG. 6). Both the ZIKV-rcCap-NS3 and rcCap-NS5 have near lethal phenotypes in C6/36 mosquito cells (FIG. 6). This indicates that recoding the ZIKV capsid coding region disrupted an RNA element that is critical for replication in mosquito cells.

These results confirm that virus genome recoding by codon optimisation works for both DENV2 and ZIKV, and was consistent in its ability to reduce DENV2 and ZIKV replication efficiency independent of cell type or animal of origin.

Recoded DENV2 and ZIKV Clones have a Small Plaque Phenotype

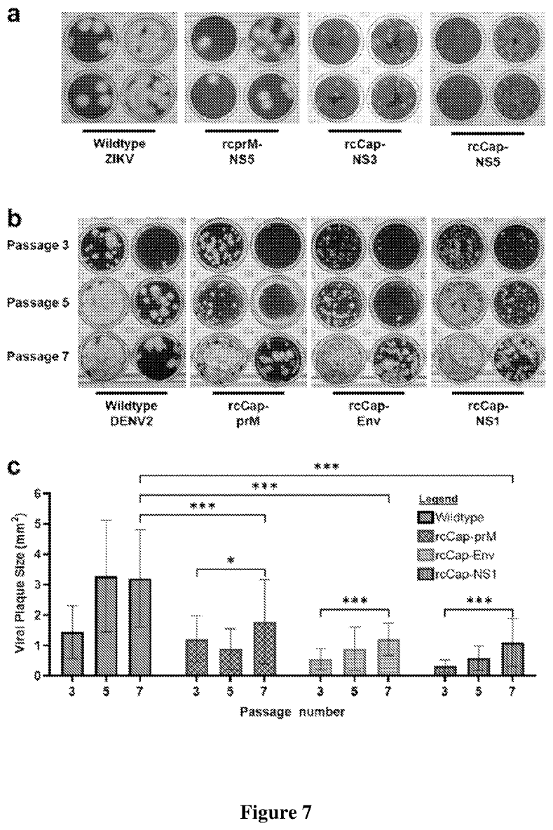

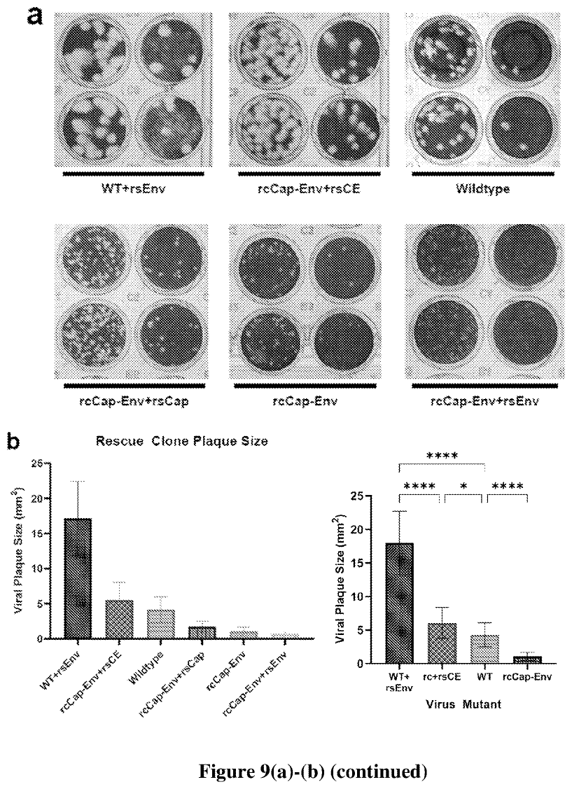

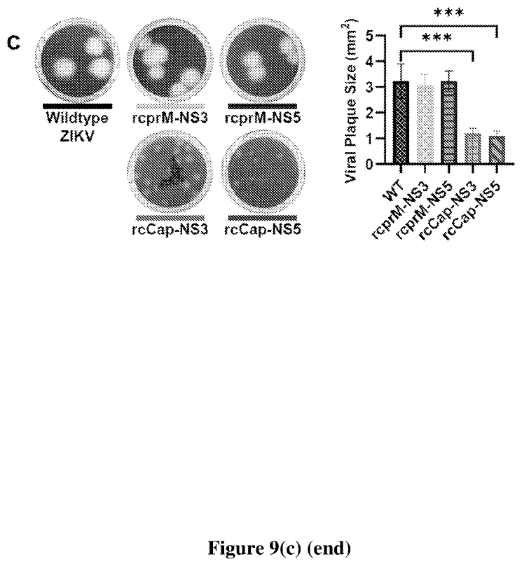

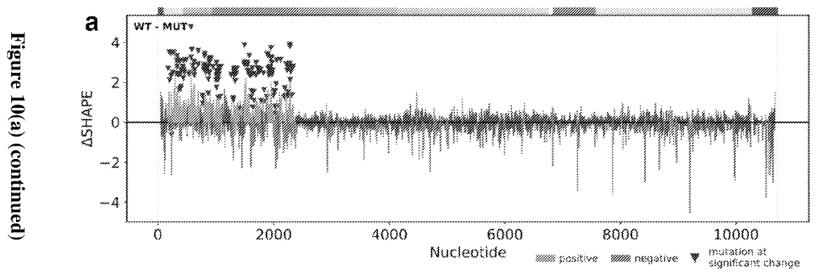

It was observed that the DENV2 rcCap-prM, rcCap-Env, and rcCap-NS1 clones as well as the ZIKV rcCap-NS3 and rcCap-NS5 clones had small plaque phenotypes (FIG. 7 and FIG. 9c). The degree of recoding generally correlated with a reduction in plaque size. However, it was also noted that the ZIKV-rcprM-NS5 clone exhibited reduced viral replication efficiency even though it did not have a small plaque phenotype.

### Genetic Stability of Wildtype and Recoded DENV2

Next, the genetic stability of the recoded DENV2 clones was tested. Wildtype DENV2 as well as the rcCap-prM, rcCap-Env, and rcCap-NS1 clones were serially passaged in BHK-21 cells. An increase in the average plaque sizes was observed for both the wildtype and recoded DENV2 clones, though the wildtype plaque sizes were still the largest (FIG. 7b & 7c). This increase in average plaque size was due to the emergence of large plaque mutants in the virus population, with the large plaque mutants becoming the dominant phenotype by passage 7. Next-generation sequencing of wildtype DENV2 as well as the rcCap-Env and rcCap-NS1 clones was performed at passage 10 to identify potential cell-line adaptation mutations, rescue mutations and reversion mutations (Tables 2 & 3).

The same a1522g (Env-M196V) and g1524a (Env-M1961) mutations were identified in both the wildtype and recoded viruses, indicating that these mutations may be cell line adaptations. The a1522g and g1524a mutations had an exceeding low co-occurrence frequency of 0.018%, meaning that once a virus acquired one of the mutations there was no more selection pressure to acquire the other.

No reversion mutations (defined as mutations which directly reverse the mutations introduced during recoding) were identified in the recoded DENV2 clones. However, potential rescue mutations (or compensatory mutations) were identified in the 5′UTR and in the capsid coding region (Table 3). The same u71g nucleotide substitution was identified in the 5′UTR of the rcCap-Env and rcCap-NS1 clones, but these occurred at low frequencies of 14.2% and 28% respectively. More importantly, a158u, u173c, and a192u nucleotide substitutions in the capsid coding region of the rcCap-Env clone, as well as a158u, c181u, and a192u mutations in the rcCap-Env clone were identified (Table 3 & 4). The a158u and a192u nucleotide substitutions had exceedingly low co-occurrence frequencies of 0.07% or less in both recoded clones, meaning that there was selection pressure for one or the other, but not both (Table 4). The nucleotide positions 158, 173, 181, and 192 are located within the DCS-PK element, which lies in the capsid coding region, immediately downstream of the 5′CS. The c181u mutation is silent, while the a158u, u173c, and a192u mutations result in capsid protein N21I, V26A, and R32S amino acid substitutions respectively (Table 3).

Predicted Structure of DCS-PK with Recoding and Rescue Mutations

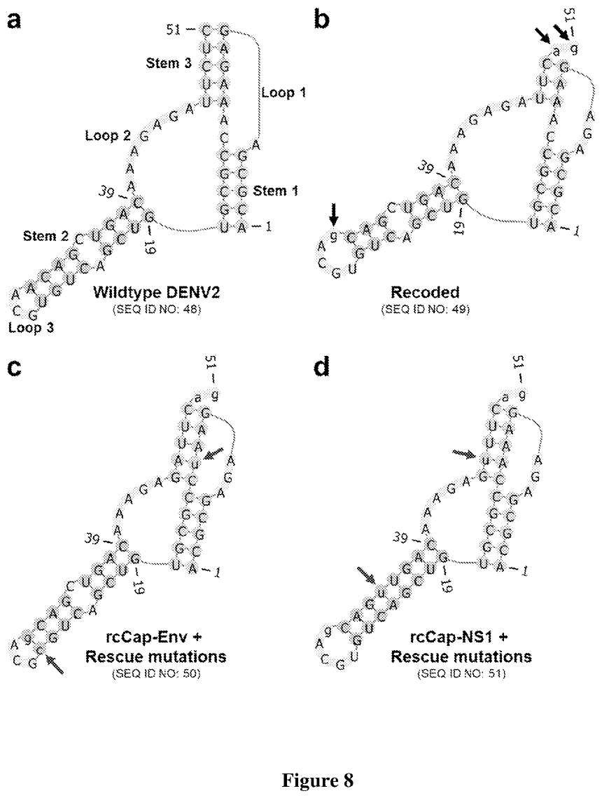

For the ‘normal’, non-reporter recoded DENV2 and ZIKV clones that do not encode EGFP, the codon optimisation starts from the 26^{th} codon of the capsid coding region (which is also the 26^{th} codon of the flavivirus polyprotein). This leaves the cHP and 5′CS intact but places the the DCS-PK element within the codon optimised region. The recoding process introduced three nucleotide substitutions into the DCS-PK sequence: a177g in stem 2, as well as u196a and c197g in stem 3 (FIG. 8a & 8b). The three recoded DENV2 clones, rcCap-prM, rcCap-Env, and rcCap-NS1, all have the same DCS-PK sequence and mutations (FIG. 8b).

The predicted DCS-PK structure of wildtype DENV2, recoded DENV2, and recoded DENV2 with the rescue mutations from rcCap-Env or rcCap-NS1, were modelled. The predicted structure confirms that the a177g mutation in stem 2, as well as the u196a and c197g mutations in stem 3, disrupt the formation of their respective stems (FIG. 8a & 8b).

The a158u, u173c, c181u, and a192u nucleotide substitution mutations were predicted to partially restore the structure of the DCS-PK, especially at stem 2 and 3 (FIGS. 8c & 8d). The a158u and a192u mutations are predicted to be gain-of-function mutations that create additional base pairings that rescue the DCS-PK stem 3. The a158u mutation can base pair with a192, but not with a192u, while the a192u mutation can base pair with a158, but not with a158u. Therefore, the a158u and a192u mutations are not compatible with each other, which would explain their exceedingly low co-occurrence frequency (Table 4).

The u173c and c181u mutations are also predicted to be gain-of-function mutations that create additional base pairings within the DCS-PK stem 2 (FIGS. 8c & 8d). The u173c mutation introduces a base pairing with a177g, which is one of the recoding mutations, while the c181u mutation introduces a base pairing with a169. Therefore, the capsid rescue mutations are gain-of-function mutations that create additional base pairings within the DCS-PK.

### Constructing Recapitulatory Rescue Clones of Recoded DENV2

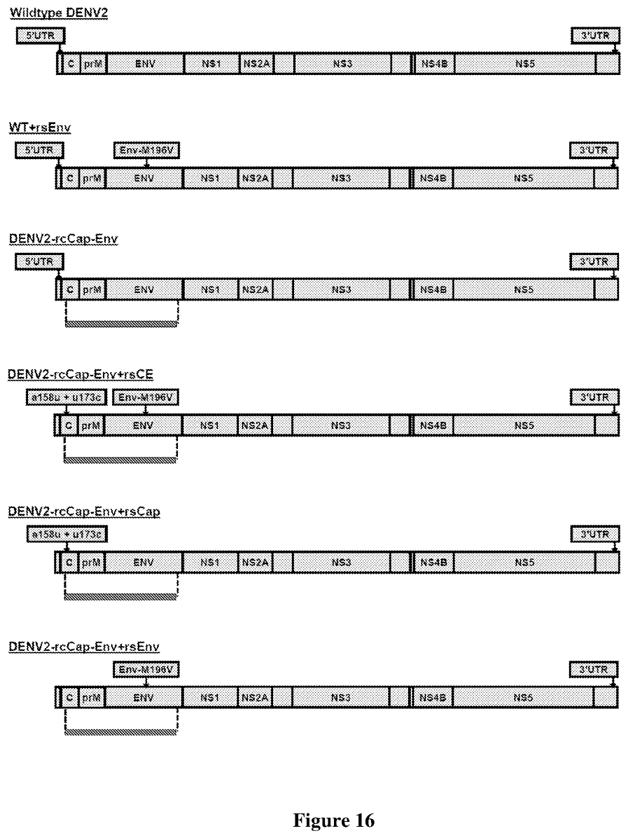

Various combinations of the rescue mutations were cloned into the rcCap-Env infectious clone (FIG. 16). The rcCap-Env clone was chosen for downstream experiments because it showed the lowest replication efficiency in human and mammalian cells. The rcCap-Env+rsCE rescue clone contains the a158u (Cap-N211), u173c (V26A), and a1522g (Env-M196V) mutations and recapitulates the dominant species of the rcCap-Env virus population at passage 10. Two additional rescue clones were also constructed to deconvolute the contributions of the capsid and envelope rescue mutations: rcCap-Env+rsCap contains the a158u (Cap-N211) and u173c (Cap-V26A) mutation while rcCap-Env+rsEnv contains only the a1522g (Env-M196V) mutation. As a control, the a1522g (Env-M196V) mutation was cloned into wildtype DENV2 to construct WT+rsEnv. WT+rsEnv recapitulates the genotype of one of the dominant species of the wildtype virus population at passage 10.

All the rescue clones were viable as they were able to form plaques (FIG. 9). Amongst all the wildtype, recoded, and rescue clones, the WT+rsEnv rescue clone that possessed the Env-M196V mutation formed the largest plaques (FIG. 9). In contrast, the rcCap-Env+rsEnv rescue clone that also possesses the Env-M196V formed the smallest plaques.

### RNA SHAPE Analysis Shows Loss of RNA Structures in Recoded DENV2

To confirm if DENV2 genome recoding disrupts genomic RNA structures, and if the disruption remained stable after serial passaging, RNA SHAPE-MaP analysis was performed on wildtype DENV2 and the rcCap-Env+rsCap rescue clone. Using the results of SHAPE analysis as a constraint, in silico modelling of wildtype and recoded virus RNA structural elements was also performed.

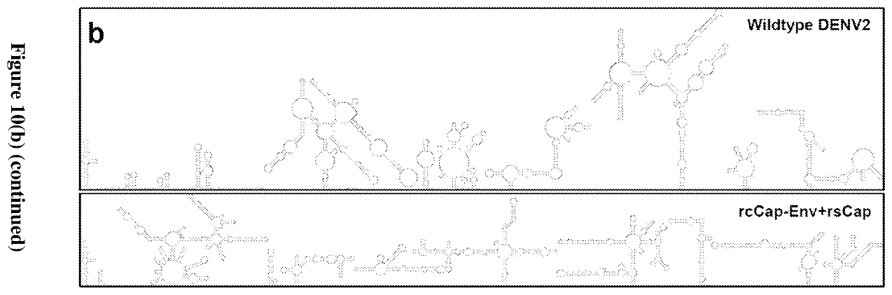

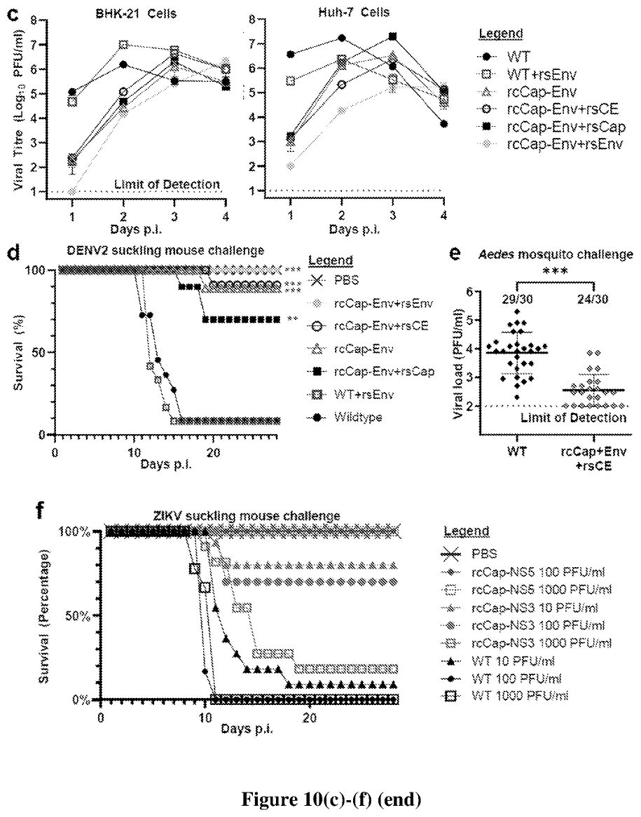

It was found that almost all the RNA structural elements that are found in the structural protein coding region of wildtype DENV2 are disrupted and no longer found in recoded DENV2 (FIG. 10a & 10b). Further, it was found that the a158u (Cap-N211) and u173c (Cap-V26A) rescue mutations did not play any meaningful role in reversing the large-scale disruptions to RNA structural elements. This confirms that DENV2 genome recoding by codon optimisation results in the disruption of potential RNA elements in the virus genome. It also indicates that these disruptions are genetically stable.

### Rescue Mutants of Recoded DENV2 Retain Delayed Viral Growth Kinetics

The rcCap-Env clone was chosen to determine if the rescue mutations were cell line adaptations because it showed the lowest replication efficiency in human and mammalian cells as well as the second lowest replication efficiency in mosquito cells. Therefore, any increase in the virus replication efficiency would be more obvious.

The a158u (Cap-N211), u173c (V26A), and a1522g (Env-M196V) mutations were cloned into the rcCap-Env clone to produce a rescue mutant, named rc+rsCE. As a control, the a1522g (Env-M196V) mutation was cloned into wildtype DENV2 to construct the WT+rsEnv mutant. It was observed that the rc+rsCE rescue mutant formed larger plaques compared to wildtype DENV2 and the rcCap-Env clone (FIG. 9). However, the WT+rsEnv mutant formed even larger plaques compared to all the other viruses (FIG. 9).

To confirm whether the mutations were cell line adaptations or rescue mutations, the viral growth kinetics of wildtype DENV2, WT+rsEnv, rcCap-Env, and rc+rsCE mutants were compared in BHK-21 and Huh-7 cells. The cells were infected at an MOI of 0.1 and viral titres were measured using plaque assay. The growth kinetics showed that the rc+rsCE rescue mutant retained the delayed growth kinetics of the parental recoded rcCap-Env clone in both BHK-21 and Huh-7 cells as their titres peaked one day later than wildtype DENV2 (FIG. 10c). When compared to the parental rc+rsCE clone, the rs+rsCE mutant had a slightly higher peak titre in BHK-21 cells, while the opposite was true in Huh-7 cells (FIG. 10c). Similar results were observed for the WT+rsEnv rescue mutant. The WT+rsEnv rescue mutant titres peaked on the same day as wildtype DENV2 in both BHK-21 and Huh-7 cells (FIG. 10c). While the WT+rsEnv mutant had a slightly higher peak titre in BHK-21 cells when compared to wildtype DENV2, the opposite was true in Huh-7 cells (FIG. 10c).

These results confirm that the Env mutation is probably a cell line adaptation. They also indicate that the capsid mutations may be cell line adaptations instead of true rescue mutations. Curiously enough, the addition of the a1522g (Env-M196V) mutation to the rcCap-Env backbone confers a further delay in replication kinetics on the resulting rcCap-Env+rsEnv rescue clone (FIG. 10c). This effect was not observed in the rcCap-Env+rsCE rescue clone with the additional capsid rescue mutations or in the wildtype backbone. Therefore, the effect of the a1522g (Env-M196V) mutation depends on whether it is cloned into a wildtype backbone or into some specific recoded backbone. This makes sense when we consider that the underlying a1522g RNA mutation can have its own effects at the functional RNA level.

### Recoded DENV2 and ZIKV Show Attenuated Neurovirulence in Suckling Mice

Finally, the in vivo attenuation of the recoded viruses was tested in a suckling mouse model. Newborn outbred white ICR mice that were less than 24 hours old were inoculated intracranially with wildtype DENV2, WT+rsEnv, rcCap-Env, or rc+rsCE clones that had been diluted to a concentration of 100,000 PFU/ml. The mice were kept for four weeks and observed daily for clinical symptoms and euthanised when they reached a humane endpoint. Both the wildtype DENV2 and WT+rsEnv mutant had similar lethality rates of 91% and 92% (n=10/11 and 11/12) respectively (FIG. 10d). In contrast, recoded rcCap-Env and its derivative rescue clones all demonstrated in vivo attenuation; the lethality rate of the rcCap-Env, +rsCE, +rsCap, and +rsEnv was significantly lower at 11%, 9%, 30%, and 0% respectively (n=1/9, 1/11, 3/10, and 0/9) (FIG. 10d). This confirms that the Env and capsid mutations are cell culture adaptations as they had no effect in vivo in the suckling mouse model of neurovirulence. These results confirm that the capsid mutations are not true rescue mutations, as they do not allow recoded DENV2 to revert to a virulent phenotype. This demonstrates that our recoding approach offers genetically stable attenuation, as serial passaging does not result in mutations that can restore virulence.

In vivo attenuation of the ZIKV-rcCap-NS3 and ZIKV-rcCap-NS5 clones were also investigated using the suckling mouse model. Newborn outbred white ICR mice that were less than 24 hours old were inoculated intracranially at a dose of 10, 100, or 1000 PFU/ml of wildtype or recoded ZIKV. Wildtype ZIKV demonstrated a high degree of virulence at doses of 10, 100, or 1000 PFU/ml, with lethality rates of 90.9%, 100%, and 100% respectively (n=1/11, 0/12, and 0/11) (FIG. 10f). In contrast, ZIKV-rcCap-NS3 demonstrated in vivo attenuation for the same three doses, with lower lethality rates of 20%, 30%, and 82% respectively (n=12/15, 7/10, and 9/11) (FIG. 10e). The ZIKV-rcCap-NS5 clone demonstrated an even higher degree of attenuation: at the higher doses of 100 or 1000 PFU/ml it had lethality rates of 0% (n=12/12 and 14/14) (FIG. 10f). This indicates that a greater degree of recoding is correlated with a greater degree of attenuation. This also demonstrates that the recoding approach can attenuate both DENV2 and ZIKV.

Recoded DENV2 Demonstrates Attenuation in Aedes albopictus Mosquitoes

Next, the rcCap-Env+rsCE clone was chosen to investigate if recoded DENV2 is attenuated in its Aedes mosquito vector, because high titres necessary for mosquito challenge could be obtained with this clone. Aedes albopictus mosquitoes were challenged with an infectious blood meal containing 2.5×10⁷ PFU/ml of either wildtype DENV2 or recoded DENV2-rcCap-Env+rsCE. The mosquitoes were kept for 11 days after oral infection, after which the plaque assay was used to determine their infection status and viral load. Compared to wildtype DENV2, recoded DENV2-rcCap-Env+rsCE was attenuated in Aedes albopictus mosquitoes. Wildtype virus was able to establish infection in 29/30 mosquitoes, whereas recoded DENV2-rcCap-Env+rsCE was only able to only infected 24/30 mosquitoes (FIG. 10e). The mosquitoes that were infected with recoded DENV2-rcCap-Env+rsCE were also found to carry a lower viral load (FIG. 10e). This demonstrates that recoded DENV2 has in vivo attenuation in both mice and mosquitoes.

### Recoding Modules for Vaccine Design

Based on the results, the flavivirus genome can be divided into four potential recoded modules (FIG. 11). The results allows prediction of how much each recoded module contributes to attenuation relative to the other modules, and how each module might function differently in human, mammalian, or mosquito cells. This gives a reliable metric for predicting the relative degree of attenuation between different recoded strains, based on the different combination of recoded modules they may possess.

Module 1 corresponds to most of the capsid coding region, starting from the 76th nucleotide (26th codon). This excludes the first 75 nucleotides of the capsid coding region. Recoded module 1 contributes the most to the loss of replication efficiency or even loss of viability in mosquito cells.

Module 2 corresponds to the premembrane and envelope coding regions. Recoded module 1 and 2 have the greatest contribution to the loss of replication efficiency in mammalian and human cells. Furthermore, recoded modules 1 and 2 by themselves are sufficient for in vivo attenuation in mice.

Module 3 corresponds to the NS1, NS2A, and NS2B coding region, as well as the first half of the NS3 coding region.

Module 4 corresponds to the latter half of the NS3 coding region, as well as the NS4A, NS4B, and NS5 coding regions. Recoded modules 3 and 4 do have a measurable impact on virus replication efficiency. However, their contribution to the overall degree of attenuation is not as strong as recoded module 1 and 2.

The RNA elements in the flavivirus genome vary greatly in sequence length, in secondary structure, and in their long-range interactions. Therefore, there will be RNA elements that do not sit neatly between the boundaries of the protein coding regions or the recoded modules that we describe below. Therefore, as discussed above, the recoding generally “spills over” across the protein coding regions. In addition to this, not all the modules may need recoding simultaneously, and there may not be a need to recode the entirety of a specific module, depending on the degree of attenuation that is desired.

Sequences for the full-length wildtype virus genomes and the recoded virus genomes, as well as the respective ORF sequences, are shown in Table 5. For most of the recoded viruses, the sequences include all four recoded modules (1 to 4).

As discussed above, the construction of an actual vaccine strain may not require the full-length recoding of all four modules. Therefore, as an example, Table 5 shows the genome sequence for DENV2-rcCap-NS5, where all four modules are fully recoded, as well as DENV2-rcCap-Env, where only modules 1 and 2 are recoded. Also shown is DENV2-rc+rsCE, the mutant containing recoded modules 1 and 2 as well as rescue mutations (or cell-line adaptations).

### Discussion

There are several potential attenuation mechanisms for synonymous virus genome recoding. One is a simple matter of codon deoptimisation leading to decreased protein translation efficiency. Another explanation is that different hosts have different dinucleotide frequency preferences, and that changes in the dinucleotide frequency triggers an innate immune responses.

Previous attempts at recoding flaviviruses utilised deoptimisation approaches, where the virus is optimised for an insect host at the expense of mammalian hosts. These served as a proof-of-concept that virus genome recoding can attenuate flaviviruses. However, the major shortcoming of this approach is that the mechanism of attenuation seems to be dependent on cell type or animal of origin. For example, deoptimised DENV2 replicates poorly in LLC-MK2 cells, but replicates normally in BHK-21 hamster cells and C6/36 mosquito cells; deoptimised DENV2 also remains transmissible by and replicates normally in Aedes mosquitoes. Similarly, deoptimised ZIKV replicates poorly in Vero monkey cells, but replicates normally in C6/36 cells.

These results are undesirable for several reasons. First, the mechanism of attenuation is inconsistent, as it can fail in BHK-21 cells even though they are also mammalian cells. Second, rather unsurprisingly, the mechanism of attenuation does not work in mosquito cells and mosquitoes. Flavivirus vaccines need to be attenuated in not only in mammalian cells, but also in insect cells in order to prevent vaccine transmission and escape by mosquito vectors.

The other recoding approach of targeting dinucleotide frequencies will potentially face the same shortcomings. Both approaches rely on mechanisms which are highly dependent on the cell line, cell type, animal of origin, the immunocompetence of an individual, or even the genetic variability that exists between individuals. These factors must be addressed if recoded viruses are to be developed into live attenuated vaccines. If the attenuation mechanism is not targeting something inherent to the virus, then the potential vaccine strain cannot be considered safe to use in humans who are immunodeficient, or who show genetic variations in their PRR pathways.

In this study, another attenuation approach is demonstrated involving synonymous virus genome recoding. It is shown that codon optimising the protein coding region of two different flaviviruses can reduce viral replication efficiency in human, mammalian, and mosquito cells. This indicates that the attenuation mechanism of this recoding approach functions in a manner that is inherent to the virus itself and that is independent of cell type or animal of origin.

The results indicate that codon optimising the DENV2 genome can enhance viral protein translation efficiency (FIG. 3b), whilst simultaneously reducing viral RNA replication efficiency (FIG. 3c). The codon optimised regions in the rcE2-90 and rcNS1 clones are short, at 90 codons and 402 codons respectively. The number of mutated codons is even smaller, at 56 codons and 252 codons respectively. The DENV2 genome encodes 3,392 codons; it is unlikely that a 50% increase in protein translation efficiency can be achieved just by increasing the favourability of 252 codons, let alone 56 codons. In a similar vein, it is unlikely that these small number of mutations in rcE2-90 and rcNS1 would have a significant impact on the overall CpG or UpA dinucleotide frequencies.

The simultaneous enhancement of viral protein translation efficiency and reduction in viral RNA replication efficiency points to an alternative mechanism, and it is likely that the codon optimisation targets RNA elements that regulate the transition of the viral RNA genome from the linear protein translation state to the circularised RNA replication state. The recoded RNA genome remains stuck in the protein translation state for longer, meaning that the RNA genome gets to spend more time expressing proteins. This is balanced out by a delayed transition to the circularised genome state, leading to a reduction in RNA replication efficiency.

RNA SHAPE analysis and in silico modelling confirmed that codon optimisation resulted in the extensive disruption of RNA structural elements in the DENV2 genome. Therefore, this approach of codon optimising the flavivirus genome can abrogate the sequence and function or RNA elements that help to regulate flavivirus RNA replication. By introducing mutations that are spread out across 58-61% of codons, this approach can target RNA elements even before they have been identified by specialised mapping techniques. This means it can be applied to any newly emerged mosquito-borne flavivirus.

The recoded DENV2 clone remains attenuated in cell culture and in vivo even after serial passage. This demonstrates that this recoding approach offers genetically stable attenuation, which is consistent with other virus genome recoding approaches.

As can be seen, the present virus genome recoding approach offers a genetically stable attenuation mechanism that targets the RNA elements that regulate RNA replication of flaviviruses such as DENV2 and ZIKV, overcoming a shortcoming of previous recoding approaches. This results in a consistent reduction in viral replication efficiency in human, mammalian, and even mosquito cells as well as in vivo attenuation in a suckling mouse model and Aedes albopictus mosquito model. Serial passaging recoded DENV2 does not result in the emergence of mutations that can rescue the delayed replication kinetics or in vivo attenuation of recoded DENV2. This demonstrates that this recoding approach confers genetically stable attenuation and has the potential to produce attenuated backbones for the development of flavivirus vaccines.

### Example 3

### Approach to Flaviviral Vaccine Development Through Genome Editing

A three-stage process may be envisioned for constructing a live attenuated flaviviral vaccine using a combination of recoded backbone mutations and accessory mutations.

In the first stage, the flavivirus genome is recoded to construct a vaccine backbone with a good baseline in vivo attenuation. This attenuation is genetically stable because each silent mutation introduced has a very minor attenuating effect and so there would be very little selection pressure for an individual mutation to revert. Because the genome recoding only introduces silent mutations, the antibody and T-cell epitope sequences of the wildtype or parent virus are preserved, thus the recoded virus is expected to retain the immunogenicity of the parent virus.

In the second stage, accessory mutations are added to the recoded genomic backbone to enhance viral attenuation and immunogenicity. Some of the accessory mutations are attenuating mutations and provide an additional layer of attenuation. The rest of the accessory mutations are immunogenic mutations that enhance the early innate immune response and early interferon response.

In the third stage, the accessory mutations are genetically stabilized by converting them to a reversion resistant form. This helps to ensure that the viral genome has genetically stable attenuation and immunogenicity.

### The NS1-G53D Mutation is a Broad-Spectrum Flavivirus Attenuating Mutation

In the second stage of viral genome modification, broad-spectrum attenuating mutations are identified that can provide an additional reserve of attenuation to the recoded viral genome.

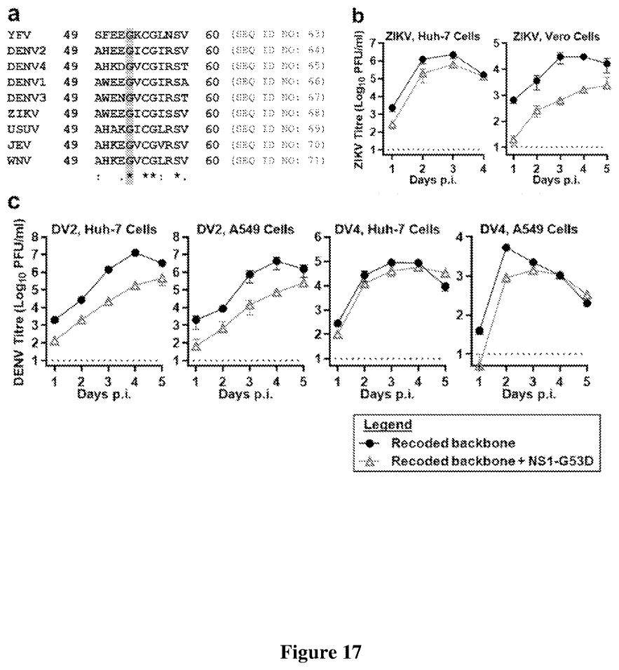

A broad-spectrum attenuating mutation was identified from the clinically validated DENV2-PDK-53 vaccine strain. One of the major attenuating mutations of the DENV2-PDK-53 vaccine strain is the NS1-G53D amino acid substitution (FIG. 17a). This mutation targets an NS1 protein ⁵³Glycine residue that is conserved across all the mosquito-borne flaviviruses (FIG. 17a).

The NS1-G53D mutation was cloned into recoded ZIKV, DENV2 and DENV4. The replication efficiencies of the recoded backbones with and without the NS1-G53D mutation were compared. It was found that addition of the NS1-G53D mutation conferred reduced replication efficiency for all the recoded viruses (FIG. 17b & 17c), therefore, the NS1-G53D mutation is compatible the attenuated recoded viral backbones, and also acts as a broad-spectrum pan-Aedes clade attenuating mutation, and potentially a pan-flavivirus attenuating mutation.

### Identifying Immunogenic Mutations

Successful flavivirus vaccines should achieve a good balance of viral attenuation and immunogenicity. The DENV2-PDK-53 NS1-G53D mutation is known to be both attenuating and immunogenic for DENV2. The dengue virus NS1 protein can directly inhibit the antiviral interferon response in a host. In contrast, the NS1-G53D enhances the host antiviral interferon response.

To investigate if the NS1-G53D mutation also acts as an immunogenic mutation for other flaviviruses, the immunogenicity of recoded DENV4 and ZIKV viruses containing the G53D mutation was analysed using the A549 human cell line as it is a well-established model for studying the antiviral innate immune response. A549 cells were infected and harvested at 24 hours post-infection for qRT-PCR analysis of genes from the early interferon response pathway. This analysis included signalling cytokines, signal transduction molecules, and antiviral effectors. Mock-infected A549 cells were used as a control and cells infected with wildtype virus were used as a positive control.

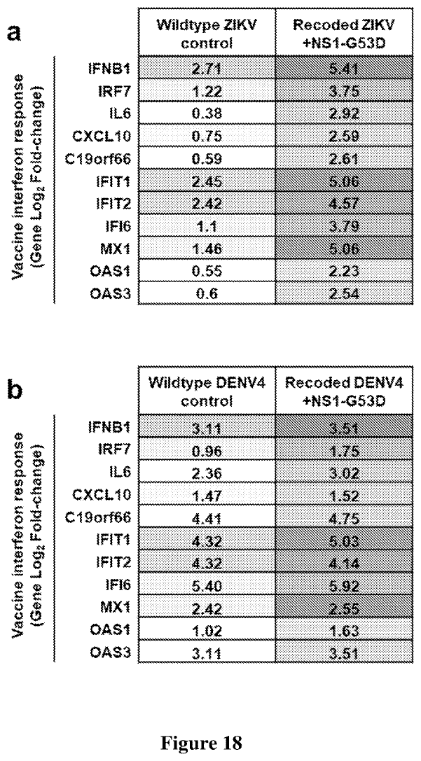

It was found that wildtype ZIKV and wildtype DENV4 could activate the early interferon response (FIG. 18). In fact, compared to wildtype ZIKV and DENV4, the recoded viruses with the NS1-G53D mutation could activate an even stronger interferon response (FIG. 18a & 18b). Therefore, the NS1-G53D can be used as a broad-spectrum immunogenic accessory mutation for the mosquito-borne flaviviruses. This enhancement of the early interferon response may correlate with better immunogenic outcomes in vaccine development.

### Genetic Stabilisation of Accessory Mutations

In the third stage, the accessory mutations are genetically stabilized. The NS1-G53D mutation starts as a single nucleotide substitution targeting the codon 53 of the NS1 protein: GGC to GaC. This results in a Gly to Asp amino acid substitution. However, this also means that a single nucleotide reversion will result in a reversion to the non-attenuating wildtype Gly residue.

For RNA viruses like flaviviruses, reversion events that only require a single nucleotide substitution occur at a very high frequency of approximately 10⁻⁴. In contrast, reversion events that require two simultaneous nucleotide substitutions will occur at an exponentially lower frequency of 10⁻⁸. Codons that require at least two simultaneous substitutions in order to revert to a non-attenuating amino acid are called reversion resistant codons.

These reversion resistant codons have two key properties. First, they encode for an attenuating amino acid. Second, any single nucleotide substitution in a reversion resistant codon will result in a codon that still encodes for an attenuating mutation. As a negative example, the original NS1-G53D mutation is not encoded by a reversion resistant codon, because an A to G single nucleotide substitution (GaC to GGC) will result in a codon that encodes for the original wildtype Gly.

To genetically stabilize the NS1-G53D mutation so that it becomes a variant encoded by a reversion resistant codon, the effects of various amino acid substitutions at the NS1-G53 position were first screened.

The following assumptions were made:
1) The starting parental codon of interest is called the primary codon.
2) The primary codon may encode for an amino acid that confers a wildtype phenotype, an attenuating phenotype, or a lethal phenotype. It may also encode a stop codon.
3) The secondary codons are the nine codons that can be derived from the primary codon via a single nucleotide substitution.
4) It is assumed that codons that encode for the same amino acid will result in the same phenotype.
5) If any particular amino acid is not characterized, it is assumed that the amino acid confers a wildtype phenotype.
6) A primary codon is reversion resistant if it satisfies two properties. First, it must encode an attenuating amino acid itself. Second, none of its secondary codons must encode an amino acid that confers a wildtype phenotype. Instead, the secondary codons should only encode stop codons or amino acids that confer attenuating or lethal phenotypes.

Additionally, to aid in automated analysis of potential reversion resistant codons, the following additional assumptions were made:
7) The primary coding space is all the potential amino acids that are encoded by the primary codon.
8) The primary score is the phenotypic score of the primary codon. It indicates that the codon encodes an amino acid that confers a wildtype phenotype, an attenuated phenotype, or a lethal phenotype.
9) The following scoring system is used in this analysis, but any similar scoring system that aids in automated analysis may be used.
10) A score of 1 indicates that the codon encodes an amino acid that confers a wildtype phenotype.
11) A score of 0 indicates that the codon encodes an amino acid that confers an attenuated phenotype.
12) A score of −0.01 indicates that the codon encodes a stop codon or an amino acid that confers a lethal phenotype.
13) The secondary coding space is all the potential amino acids and stop codons that are encoded by the secondary codons.
14) The secondary score is the sum of the phenotypic scores of the secondary codons.
15) Codons that encode the same amino acid are assumed to give the same phenotype and thus have the same primary score.
16) A primary codon is a reversion resistant codon if it satisfies two properties. First, its primary score is exactly 0. Second, the secondary score of its secondary codons is 0 or less (N≤0).
17) A primary codon with a primary score of 1 or −0.01 confers a wildtype or lethal phenotype respectively, and is therefore not a useful starting codon for mutagenesis.

A phenotypic screen was conducted on the NS1-G53 position. To ensure that results are broadly applicable to other flaviviruses, the screen was performed in ZIKV, the outlier flavivirus of the Aedes clade. The NS1-G53D mutation confers a small-plaque phenotype on ZIKV, thus the small-plaque phenotype was used as a marker of attenuation for the NS1-G53 position. A total of 12 amino acid substitutions were screened. It was found that all 12 amino acid substitutions resulted in an attenuated or lethal phenotype (Table 6). As discussed above, amino acid that were not tested were assumed to confer a wildtype phenotype.

Next, to screen for reversion resistant codons for the NS1-G53 position, an automated scoring method was used to calculate the secondary scores of all 64 potential codons for the NS1-G53 position. These are summarised in Table 7.

Nine secondary codons were shortlisted that had secondary scores of 0 or less (Table 8). Of these nine codons, seven encoded for amino acids that conferred lethal phenotypes and are thus not useful for live vaccine development. The remaining two codons could be used as reversion resistant codons: TCA, which encodes for serine, and ATG, which encodes for methionine.

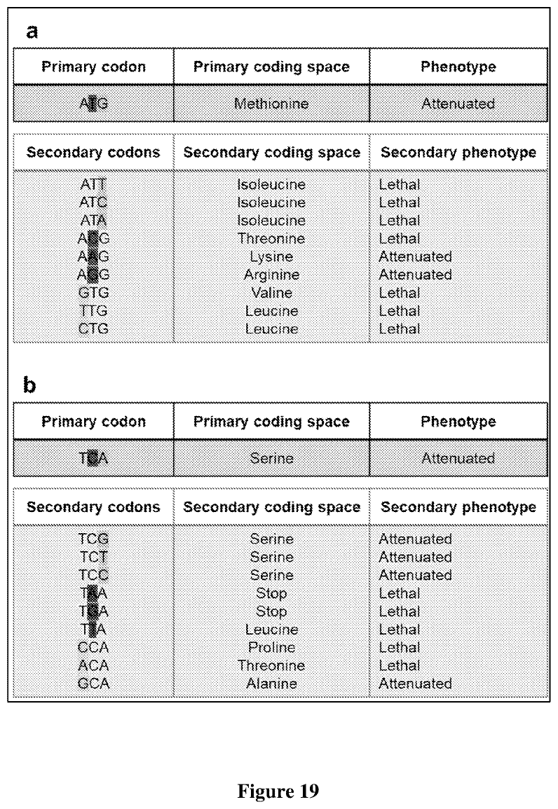

Both serine and methionine confer an attenuated phenotype. Further, all the secondary codons of TCA and ATG encode stop codons, attenuating mutations, or lethal mutations (FIG. 19). Therefore, TCA and ATG are reversion resistant codons for the NS1-G53 position.

### Summary

Using a combination of recoded backbone mutations and accessory mutations, genetically stable live attenuated flaviviral vaccine may be constructed that are attenuated and immunogenic. These modified viruses retain their original antigenic epitopes, which ensures that they can generate virus- or serotype-specific antibody and T-cell responses that will confer protection against infection without generating antibody dependent enhancement. This vaccine development approach can potentially be applied to any mosquito-borne flavivirus to speed up the development of flavivirus vaccines.

TABLE 2
Detailed description of recoded DENV2 and ZIKV clones. The regions of the
genome that were codon optimised are described in detail. Total number
of codons that lie within these regions, as well as the number of codons
that were mutated with silent mutations are also listed. The percentage
of affected codons is calculated from the region targeted for recoding.

| | | | Total | No. of | % of |
|---|---|---|---|---|---|
| | | Recoded region | no. of | mutated | affected |
| Virus | Clone | of genome | Codons | Codons | codons |
| DENV2 | rcE2 | Codons 201 to 470 of Env | 270 | 172 | 63.70% |
| DENV2 | rcE2-90 | Codons 381 to 470 of Env | 90 | 56 | 62.22% |
| DENV2 | rcE2-60 | Codons 411 to 470 of Env | 60 | 39 | 65% |
| DENV2 | rcE2-50 | Codons 421 to 470 of Env | 50 | 32 | 64% |
| DENV2 | rcE2-40 | Codons 431 to 470 of Env | 40 | 27 | 67.5% |
| DENV2 | rcNS1 | From the last 25 codons of Env | 402 | 252 | 62.69% |
| | | to the first 25 codons of NS2A | | | |
| DENV2 | rcCap-prM | From the 26th codon of Capsid | 279 | 165 | 59.14% |
| | | to the first 25 codons of Env | | | |
| DENV2 | rcCap-Env | From the 26th codon of Capsid | 725 | 445 | 61.38% |
| | | to the 470th codon of Env, | | | |
| | | last 25 codons of Env | | | |
| | | remain untouched | | | |
| DENV2 | rcCap-NS1 | From the 26th codon of Capsid | 1127 | 697 | 61.85% |
| | | to the first 25 codons of NS2A | | | |
| ZIKV | rcprM-NS3 | From the last 25 codons of Capsid | 1623 | 951 | 58.59% |
| | | to the first 218 codons of NS3. | | | |
| | | Stops at a BsiWI restriction site | | | |
| ZIKV | rcprM-NS5 | From the last 25 codons of Capsid | 2549 | 1474 | 57.83% |
| | | to the first 126 codons of NS5. | | | |
| | | Stops at an AflII restriction site | | | |
| ZIKV | rcCap-NS3 | From the 26^{th} codon of Capsid | 1695 | 993 | 58.58% |
| | | to the first 218 codons of NS3. | | | |
| | | Stops at a BsiWI restriction site | | | |
| ZIKV | rcCap-NS5 | From the 26^{th} codon of Capsid | 2621 | 1516 | 57.84% |
| | | to the first 126 codons of NS5. | | | |
| | | Stops at an AflII restriction site | | | |

TABLE 3
Analysis of next-generation sequencing of wildtype and recoded DENV2 after serial
passage. Relative frequencies of mutations are shown for each nucleotide position.

| Nucleotide | Region of | Nucleotide | Amino acid | Frequency in virus | | |
|---|---|---|---|---|---|---|
| Position | Genome | substitution | substitution | Wildtype | rcCap-Env | rcCap-NS1 |
| 71 | 5′UTR | u to g | NA | 0% | 14.2% | 28% |
| 158 | Capsid | a to u | Asn to Ile | 0% | 60.7% | 21.6% |
| 173 | Capsid | u to c | Val to Ala | 0% | 52.5% | 0% |
| 181 | Capsid | c to u | Leu to Leu | 0% | 0% | 76% |
| 192 | Capsid | a to u | Arg to Ser | 0% | 39.4% | 79.3% |
| 1522 | Env | a to g | Met to Val | 31.2% | 57.4% | 22.1% |
| 1524 | Env | g to a | Met to Ile | 37.4% | 0% | 58.4% |

TABLE 4
Frequencies of co-occurrence of rescue mutations in the capsid coding region
of (a) rcCap-Env and (b) rcCap-NS1. Rescue mutations in bold and underlined.

| | | Nucleotide | | | | | | | |
|---|---|---|---|---|---|---|---|---|---|
| (a) Nucleotide position in | 158 | a | a | a | a | u | u | u | u |
| rcCap-Env | 173 | u | c | u | c | u | c | u | c |
| | 192 | a | a | u | u | a | a | u | u |
| Co-occurrence frequency (%) | | 0 | 0.04 | 0.02 | 38.6 | 0.01 | 61.3 | 0 | 0.01 |
| (b) Nucleotide position | 158 | a | a | a | a | u | u | u | u |
| in rcCap-NS1 | 181 | c | u | c | u | c | u | c | u |
| | 192 | a | a | u | u | a | a | u | u |

| | | | | | | | | |
|---|---|---|---|---|---|---|---|---|
| Co-occurrence frequency (%) | 0.03 | 0.03 | 3.44 | 75.4 | 20.7 | 0.3 | 0.04 | 0.03 |

TABLE 5
Full-length sequences and sequences of the open reading
frame (ORF) of modified flaviviruses of this disclosure.

| | | Full length | | |
|---|---|---|---|---|
| | | sequence: | | |
| | | Inclusive of 5′ | | |
| | | UTR and 3′ UTR | ORF | |
| Virus | Clone | sequence | sequence | Comments |
| Dengue virus type | Wildtype | SEQ ID NO: 1 | SEQ ID NO: 2 | Wildtype virus |
| 2 strain 16681 | | | | |
| Dengue virus type | rcCap-NS5 | SEQ ID NO: 3 | SEQ ID NO: 4 | Full recoding of |
| 2 strain 16681 | (fully | | | modules 1 to 4 |
| | recoded) | | | |
| Dengue virus type | rcCap-Env | SEQ ID NO: 5 | SEQ ID NO: 6 | Recoding of modules |
| 2 strain 16681 | | | | 1 and 2 |
| Dengue virus type | rcCap- | SEQ ID NO: 7 | SEQ ID NO: 8 | Recoding of modules |
| 2 strain 16681 | Env+rsCE | | | 1 and 2 |
| Zika virus strain | Wildtype | SEQ ID NO: 9 | SEQ ID NO: 10 | Wildtype virus |
| PRVABC59 | | | | |
| Zika virus strain | rcCap-NS3 | SEQ ID NO: 11 | SEQ ID NO: 12 | Recoding of modules |
| PRVABC59 | | | | 1 to 3 |
| Zika virus strain | Fully | SEQ ID NO: 13 | SEQ ID NO: 14 | Full recoding of |
| PRVABC59 | recoded | | | modules 1 to 4 |
| Dengue virus type | Wildtype | SEQ ID NO: 15 | SEQ ID NO: 16 | Wildtype virus |
| 1 strain West | | | | |
| Pacific 74 | | | | |
| Dengue virus type | rcCap-NS3 | SEQ ID NO: 17 | SEQ ID NO: 18 | Recoding of modules |
| 1 strain West | | | | 1 to 3 |
| Pacific 74 | | | | |
| Dengue virus type | Fully | SEQ ID NO: 19 | SEQ ID NO: 20 | Full recoding of |
| 1 strain West | recoded | | | modules 1 to 4 |
| Pacific 74 | | | | |
| Dengue virus type | Wildtype | SEQ ID NO: 21 | SEQ ID NO: 22 | Wildtype virus |
| 3 strain C0360/94 | | | | |
| Dengue virus type | rcCap-NS3 | SEQ ID NO: 23 | SEQ ID NO: 24 | Recoding of modules |
| 3 strain C0360/94 | | | | 1 to 3 |
| Dengue virus type | Fully | SEQ ID NO: 25 | SEQ ID NO: 26 | Full recoding of |
| 3 strain C0360/94 | recoded | | | modules 1 to 4 |
| Dengue virus type | Wildtype | SEQ ID NO: 27 | SEQ ID NO: 28 | Wildtype virus |
| 4 strain | | | | |
| EHI310A129SY10 | | | | |
| Dengue virus type | rcCap-NS3 | SEQ ID NO: 29 | SEQ ID NO: 30 | Recoding of modules |
| 4 strain | | | | 1 to 3 |
| EHI310A129SY10 | | | | |
| Dengue virus type | Fully | SEQ ID NO: 31 | SEQ ID NO: 32 | Full recoding of |
| 4 strain | recoded | | | modules 1 to 4 |
| EHI310A129SY10 | | | | |
| Kunjin virus | Wildtype | SEQ ID NO: 33 | SEQ ID NO: 34 | Wildtype virus |
| Kunjin virus | rcCap-NS3 | SEQ ID NO: 35 | SEQ ID NO: 36 | Recoding of modules |
| | | | | 1 to 3 |
| Kunjin virus | Fully | SEQ ID NO: 37 | SEQ ID NO: 38 | Full recoding of |
| | recoded | | | modules 1 to 4 |
| Dengue virus type | Wildtype | SEQ ID NO: 39 | SEQ ID NO: 40 | Wildtype virus |
| 3 strain | | | | |
| EHIE08901Y19 | | | | |
| Dengue virus type | rcCap-NS3 | SEQ ID NO: 41 | SEQ ID NO: 42 | Recoding of modules |
| 3 strain | | | | 1 to 3 |
| EHIE08901Y19 | | | | |
| Dengue virus type | Fully | SEQ ID NO: 43 | SEQ ID NO: 44 | Full recoding of |
| 3 strain | recoded | | | modules 1 to 4 |
| EHIE08901Y19 | | | | |
| Dengue virus type | Fully | SEQ ID NO: 72 | SEQ ID NO: 73 | Full recoding of |
| 2 strain 16681 | recoded + | | | module 1 to 4, with |
| | NS1- | | | genetically stabilised |
| | G53S- | | | NS1-G53S (GGC to |
| | TCA | | | TCA) accessory |
| | | | | mutation |
| Zika virus strain | Fully | SEQ ID NO: 74 | SEQ ID NO: 75 | Full recoding of |
| PRVABC59 | recoded + | | | module 1 to 4, with |
| | NS1- | | | genetically stabilised |
| | G53S- | | | NS1-G53S (GGC to |
| | TCA | | | TCA) accessory |
| | | | | mutation |
| Dengue virus type | Fully | SEQ ID NO: 76 | SEQ ID NO: 77 | Full recoding of |
| 1 strain West | recoded + | | | module 1 to 4, with |
| Pacific 74 | NS1- | | | genetically stabilised |
| | G53S- | | | NS1-G53S (GGC to |
| | TCA | | | TCA) accessory |
| | | | | mutation |
| Dengue virus type | Fully | SEQ ID NO: 78 | SEQ ID NO: 79 | Full recoding of |
| 3 strain C0360/94 | recoded + | | | module 1 to 4, with |
| | NS1- | | | genetically stabilised |
| | G53S- | | | NS1-G53S (GGC to |
| | TCA | | | TCA) accessory |
| | | | | mutation |
| Dengue virus type | Fully | SEQ ID NO: 80 | SEQ ID NO: 81 | Full recoding of |
| 4 strain | recoded + | | | module 1 to 4, with |
| EHI310A129SY10 | NS1- | | | genetically stabilised |
| | G53S- | | | NS1-G53S (GGC to |
| | TCA | | | TCA) accessory |
| | | | | mutation |
| Kunjin virus | Fully | SEQ ID NO: 82 | SEQ ID NO: 83 | Full recoding of |
| | recoded + | | | module 1 to 4, with |
| | NS1- | | | genetically stabilised |
| | G53S- | | | NS1-G53S (GGC to |
| | TCA | | | TCA) accessory |
| | | | | mutation |
| dengue virus type | Fully | SEQ ID NO: 84 | SEQ ID NO: 85 | Full recoding of |
| 3 strain | recoded + | | | module 1 to 4, with |
| EHIE08901Y19 | NS1- | | | genetically stabilised |
| | G53S- | | | NS1-G53S (GGC to |
| | TCA | | | TCA) accessory |
| | | | | mutation |

TABLE 6
Characterisation of amino acid phenotypes
at the NS1-G53 position.

| Amino acid | Phenotype | Primary Score |
|---|---|---|
| Alanine | Attenuated | 0 |
| Arginine | Attenuated | 0 |
| Asparagine | Not tested, assumed | 1 |
| | to be wildtype | |
| Aspartic acid | Attenuated | 0 |
| Cysteine | Not tested, assumed | 1 |
| | to be wildtype | |
| Glutamine | Attenuated | 0 |
| Glutamic acid | Not tested, assumed | 1 |
| | to be wildtype | |
| Glycine | Wildtype | 1 |
| Histidine | Not tested, assumed | 1 |
| | to be wildtype | |
| Isoleucine | Lethal | −0.01 |
| Leucine | Lethal | −0.01 |
| Lysine | Attenuated | 0 |
| Methionine | Attenuated | 0 |
| Phenylalanine | Not tested, assumed | 1 |
| | to be wildtype | |
| Proline | Lethal | −0.01 |
| Serine | Attenuated | 0 |
| Threonine | Lethal | −0.01 |
| Tryptophan | Not tested, assumed | 1 |
| | to be wildtype | |
| Tyrosine | Not tested, assumed | 1 |
| | to be wildtype | |
| Valine | Lethal | −0.01 |
| Stop codon | Essentially the same | −0.01 |
| | as lethal | |

TABLE 7
Summary of primary and secondary scores for all
64 potential codons at the NS1-G53 position.

| | | Amino | Primary | Secondary |
|---|---|---|---|---|
| No. | Codon | Acid | Score | Score |
| 1 | TTT | Phenylalanine | 1 | 2.95 |
| 2 | TTC | Phenylalanine | 1 | 2.95 |
| 3 | TTA | Leucine | −0.01 | 1.94 |
| 4 | TTG | Leucine | −0.01 | 2.96 |
| 5 | TCT | Serine | 0 | 2.98 |
| 6 | TCC | Serine | 0 | 2.98 |
| 7 | TCA | Serine | 0 | −0.05 |
| 8 | TCG | Serine | 0 | 0.96 |
| 9 | TAT | Tyrosine | 1 | 4.98 |
| 10 | TAC | Tyrosine | 1 | 4.98 |
| 11 | TAA | Stop | −0.01 | 2.97 |
| 12 | TAG | Stop | −0.01 | 3.98 |
| 13 | TGT | Cysteine | 1 | 4.99 |
| 14 | TGC | Cysteine | 1 | 4.99 |
| 15 | TGA | Stop | −0.01 | 3.98 |
| 16 | TGG | Tryptophan | 1 | 2.97 |
| 17 | CTT | Leucine | −0.01 | 1.94 |
| 18 | CTC | Leucine | −0.01 | 1.94 |
| 19 | CTA | Leucine | −0.01 | −0.07 |
| 20 | CTG | Leucine | −0.01 | −0.06 |
| 21 | CCT | Proline | −0.01 | 0.95 |
| 22 | CCC | Proline | −0.01 | 0.95 |
| 23 | CCA | Proline | −0.01 | −0.05 |
| 24 | CCG | Proline | −0.01 | −0.05 |
| 25 | CAT | Histidine | 1 | 2.98 |
| 26 | CAC | Histidine | 1 | 2.98 |
| 27 | CAA | Glutamine | 0 | 2.97 |
| 28 | CAG | Glutamine | 0 | 2.97 |
| 29 | CGT | Arginine | 0 | 2.98 |
| 30 | CGC | Arginine | 0 | 2.98 |
| 31 | CGA | Arginine | 0 | 0.97 |
| 32 | CGG | Arginine | 0 | 1.98 |
| 33 | GTT | Valine | −0.01 | 1.95 |
| 34 | GTC | Valine | −0.01 | 1.95 |
| 35 | GTA | Valine | −0.01 | 1.94 |
| 36 | GTG | Valine | −0.01 | 1.95 |
| 37 | GCT | Alanine | 0 | 0.97 |
| 38 | GCC | Alanine | 0 | 0.97 |
| 39 | GCA | Alanine | 0 | 1.97 |
| 40 | GCG | Alanine | 0 | 1.97 |
| 41 | GAT | Aspartic acid | 0 | 5.99 |
| 42 | GAC | Aspartic acid | 0 | 5.99 |
| 43 | GAA | Glutamic acid | 1 | 1.98 |
| 44 | GAG | Glutamic acid | 1 | 1.98 |
| 45 | GGT | Glycine | 1 | 3.99 |
| 46 | GGC | Glycine | 1 | 3.99 |
| 47 | GGA | Glycine | 1 | 3.98 |
| 48 | GGG | Glycine | 1 | 4.99 |
| 49 | ATT | Isoleucine | −0.01 | 1.95 |
| 50 | ATC | Isoleucine | −0.01 | 1.95 |
| 51 | ATA | Isoleucine | −0.01 | −0.06 |
| 52 | ATG | Methionine | 0 | −0.07 |
| 53 | ACT | Threonine | −0.01 | 0.95 |
| 54 | ACC | Threonine | −0.01 | 0.95 |
| 55 | ACA | Threonine | −0.01 | −0.05 |
| 56 | ACG | Threonine | −0.01 | −0.04 |
| 57 | AAT | Asparagine | 1 | 2.98 |
| 58 | AAC | Asparagine | 1 | 2.98 |
| 59 | AAA | Lysine | 0 | 2.97 |
| 60 | AAG | Lysine | 0 | 2.98 |
| 61 | AGT | Serine | 0 | 2.98 |
| 62 | AGC | Serine | 0 | 2.98 |
| 63 | AGA | Arginine | 0 | 0.97 |
| 64 | AGG | Arginine | 0 | 1.99 |

TABLE 8
Summary of primary and secondary scores for shortlisted
codons at the NS1-G53 position. All of these codons
have a secondary score of 0 or less.

| | | Amino | Primary | Secondary |
|---|---|---|---|---|
| No. | Codon | Acid | Score | Score |
| 7 | TCA | Serine | 0 | −0.05 |
| 19 | CTA | Leucine | −0.01 | −0.07 |
| 20 | CTG | Leucine | −0.01 | −0.06 |
| 23 | CCA | Proline | −0.01 | −0.05 |
| 24 | CCG | Proline | −0.01 | −0.05 |
| 51 | ATA | Isoleucine | −0.01 | −0.06 |
| 52 | ATG | Methionine | 0 | −0.07 |
| 55 | ACA | Threonine | −0.01 | −0.05 |
| 56 | ACG | Threonine | −0.01 | −0.04 |

1.-27. (canceled)

28. A method of attenuating a flavivirus, the method comprising modifying the viral genome to have a protein coding sequence having one or more synonymous codons as compared to the protein coding sequence of a parent virus, wherein the modified viral genome is associated with increased protein expression as compared to the viral genome of the parent virus in a mammalian host.

29. The method of claim 28, wherein the one or more synonymous codons is predicted or found to lead to increased protein expression as compared to the viral genome of the parent virus in a mammalian host.

30. The method of claim 28, wherein the modified viral genome is also associated with decreased protein expression in a non-mammalian host as compared to the viral genome of the parent virus, optionally wherein the non-mammalian host is an arthropod, bird or shell fish.

31. The method of claim 28, wherein the mammalian host is a human, optionally wherein the non-mammalian host is an arthropod, bird or shell fish.

32. The method of claim 30, wherein the method attenuates replication of the flavivirus in both the mammalian host and the non-mammalian host.

33. The method of claim 28, wherein the one or more synonymous codons disrupts at least one RNA element involved in viral replication, optionally wherein the at least one RNA element comprises the downstream of 5′ cyclization sequence pseudoknot (DCS-PK).

34. The method of claim 28, wherein the method modifies the viral genome to have a protein coding sequence having at least 50% of synonymous codons.

35. The method of claim 28, wherein the protein coding sequence is the flaviviral open reading frame (ORF), and wherein the method modifies the ORF to have at least 20% of synonymous codons.

36. The method of claim 28, wherein the synonymous codons are randomly distributed within the protein coding sequence or a portion of the protein coding sequence.

37. The method of claim 36, wherein the portion of the protein coding sequence comprises or consists of the coding sequence or a fragment thereof of the capsid protein (C), premembrane protein (prM), envelop protein (E), non-structural (NS) protein 1 (NS1), NS2A protein, NS2B protein, NS3 protein, NS4A protein, NS4B protein or NS5 protein, or a combination thereof.

38. The method of claim 37, wherein the synonymous codons are randomly distributed within the portion of the protein coding sequence that comprises or consists of:
the C coding sequence or a fragment thereof;
the C, prM and E coding sequences, or a fragment thereof;
the C, prM, E, NS1, NS2A, NS2B and NS3 coding sequences, or a fragment thereof; or
the C, prM, E, NS1, NS2A, NS2B, NS3, NS4A, NS4B and NS5 coding regions, or a fragment thereof;
optionally wherein i) the synonymous codons are randomly distributed within the portion of the protein coding sequence that comprises or consists of the C, prM and E coding sequences, or ii) a fragment thereof or the synonymous codons are randomly distributed within the portion of the protein coding sequence that comprises or consists of the C, prM, E, NS1, NS2A, NS2B and NS3 coding sequences, or a fragment thereof.

39. The method of claim 28, wherein the method modifies the viral genome to have a protein coding sequence having at least 70% sequence identity to SEQ ID NO: 6, 4, 8, 12, 14, 18, 20, 24, 26, 30, 32, 36, 38, 42 or 44.

40. The method of claim 28, wherein the method further comprises modifying codon 53 of the NS1 gene to code for alanine (A), arginine (R), aspartic acid (D), glutamine (N), lysine (K), methionine (M) or serine (S).

41. The method of claim 39, wherein codon 53 of the NS1 gene is modified to TCA or ATG.

42. A modified flavivirus obtained according to the method of claim 28.

43. A modified flavivirus comprising a viral genome comprising a modified virus protein coding sequence having one or more synonymous codons as compared to the protein coding sequence of a parent virus, wherein the modified viral genome is associated with increased protein expression as compared to the viral genome of the parent virus in a mammalian host.

44. The modified flavivirus of claim 43, wherein the modified flavivirus is an attenuated flavivirus.

45. The modified flavivirus of claim 43, wherein the modified flavivirus comprises a protein coding sequence having at least 70% sequence identity to SEQ ID NO: 6, 4, 8, 12, 14, 18, 20, 24, 26, 30, 32, 36, 38, 42 or 44.
